(19) United States
(12) Patent Application Publication
(10) Pub. No.: US 2005/0048463 A1
(43) Pub. Date: Mar. 3, 2005

(54) Systems and methods for determining cell type composition of mixed cell populations using gene expression signatures

(76) Inventors: David Xing-Fei Deng, Mountain View, CA (US); Anya Tsalenko, Chicago, IL (US); Zohar Yakhini, Ramat Hasharon (IL); Laurakay Bruhn, Mountain View, CA (US); Amir Ben-Dor, Bellevue, WA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/841,164

(22) Filed: May 7, 2004

Related U.S. Application Data
(60) Provisional application No. 60/468,848, filed on May 7, 2003.

(51) Int. Cl. C12Q 1/70; C12Q 1/68

(52) U.S. Cl. 435/4; 435/6

(57) ABSTRACT

The present invention provides systems and methods for determining the cell type composition of a mixed cell population. The invention provides systems and methods for identifying and defining pure cell type specific signatures. These pure cell type specific signatures may be used to determine the cell type composition of a mixed cell population. The systems and methods of the invention may be used for a variety of research and clinical purposes. For example, they may be used to detect the presence or absence of cells of particular types, and to determine whether variations in gene expression, e.g., between different samples, represent true changes in gene expression or differences in cell type composition of the samples.

### CROSS-REFERENCE TO RELATED APPLICATIONS

This application claims priority to U.S. Provisional Patent Application Ser. No. 60/468,848, filed May 7, 2003. The contents of the foregoing application are incorporated herein by reference.

### BACKGROUND OF THE INVENTION

The advent of technologies capable of detecting and quantifying gene expression has contributed greatly to the understanding of differences between cell types at a molecular level. Measurement of RNA (e.g., using Northern blots) and protein (e.g., using a variety of immunological techniques) has led to the identification of numerous molecular markers, whose presence, absence, or relative level may be used to characterize cells and classify them as belonging to particular types. Thus the concept of phenotype has broadened considerably beyond the various morphological characteristics that were traditionally used to distinguish different cell types.

While methods such as Northern and Western blots are generally limited to measurement of a few or at most a few dozen genes or proteins, gene expression profiling using microarray technology offers the opportunity to rapidly and efficiently quantify gene expression patterns of over thousands of genes. Gene expression profiling has been applied to a large number of different cell types. For example, gene expression profiling has been used to investigate systematic variations in gene expression patterns in a set of human cancer cell lines (Ross, D., et al., Nat Genet, 24(3):227-35, 2000). These experiments identified certain genes that were more highly expressed in certain cell types than in others. Attempts to use gene expression profiling to distinguish between diseased cells and their normal counterparts and to distinguish between subtypes of a particular disease have also been made. For example, gene expression profiling has been used to compare normal breast tissue with breast cancer tissue (Perou, C., et al., Proc Natl Acad Sci USA 96(16), 1999:9212-7). Gene expression profiling has also been used in attempts to classify breast tumors (Perou, C., et al., Nature, 406(6797):747-52, 2000) and lymphomas (Alizadeh, A., et al., Nature, 403(6769):503-11, 2000), and to analyze various other tumor types;

Although experiments such as those mentioned above may help to identify genes whose expression is associated with disease, approaches employed thus far suffer from a number of shortcomings. For example, many biological phenomena of interest, including manifestations of various diseases and physiological states, occur in settings where multiple cell types are present. Generally it may not be possible to isolate pure populations of cells for analysis. Thus many clinical samples such as biopsy samples include a mixture of different cell types, and the proportions of different cell types varies between samples. In such settings the existence of cell type specific gene expression patterns may be easily obscured, which may make the data difficult to interpret. The present invention provides systems and methods for analyzing mixed cell populations, thereby addressing some of these limitations.

### SUMMARY OF THE INVENTION

The present invention provides systems and methods for determining the cell type or cell state composition of a mixed cell population. The invention provides systems and methods for identifying and defining pure cell type and pure cell state specific signatures. These pure cell type or cell state specific signatures may be used for a variety of purposes, e.g., to determine the cell type or cell state composition of mixed cell populations, to detect the presence or absence of cells of particular types or in particular states, and to determine whether variations in measured gene expression, e.g., between different samples, represent true changes in gene expression or differences in cell type or cell state composition of the samples.

In one aspect, the invention provides a method of analyzing a cell population comprising the step of quantitatively determining the cell type or cell state composition of the cell population. According to certain embodiments of the invention the cell population is a mixed cell population, wherein the mixed cell population has a cell composition including at least two cell types or cell states, and the method comprises the step of quantitatively determining the cell type or cell state composition of the mixed cell population. Thus the invention provides a method of analyzing a mixed cell population comprising the steps of: (i) providing or determining a pure cell type or pure cell state signature for cells of different cell types or states in the mixed cell population; and (ii) quantitatively determining the number, proportion, or relative number of cells of different cell types or cell states in the mixed cell population using the pure cell type or pure cell state signatures for the cell types or cell states. According to certain embodiments of the invention the step of solving comprises solving a matrix equation that relates the pure cell type or pure cell state signatures to gene expression levels measured in the mixed cell population. The pure cell type or pure cell state signature of a cell type or cell state generally comprises the expression level of each of a set of genes in cells of that type or state, and according to the inventive methods the expression level of these genes is measured in the mixed cell population for the purpose of determining the composition of the mixed cell population.

According to certain embodiments of the invention the mixed cell population contains a number of cells of at least two cell types or at least two cell states, and the step of quantitatively determining the cell type or cell state composition comprises steps of: (i) obtaining an expression profile for the mixed cell population over a set of genes; and (ii) computing an approximate solution for one or more elements in a vector q, where q is a vector of quantities representing the number or proportion of cells of each type or state present in the mixed cell population, and wherein q satisfies the matrix equation Pq=m, where P is a matrix of pure cell type or cell state signatures and m is a vector of quantities including mixed cell population expression levels of genes. According to certain embodiments of the invention the number of cells is expressed in terms of a unit quantity of cells.

As used herein, determining the concept of “cell type” is understood to include cells that have the same embryological origin but that may differ phenotypically, e.g., due to any of a number of reasons. For example, the cells may be at different stages along a developmental pathway, or in different physiological states due to environmental conditions, stimuli, disease, etc. It will be appreciated that the distinction between “cell type” and “cell state” may be somewhat arbitrary. For example, two populations of cells that are initially identical or substantially identical in phenotype, e.g., two populations of mature T cells, may be considered to be of the same cell type and in the same cell state. If one population is exposed to an antigen that binds to the T cell receptor, the population will become activated and will exhibit changes in expression profile. The two populations may then be considered to constitute different cell types or different cell states. In general, the methods of the invention may be applied in an identical manner regardless of whether populations of cells are considered to be of different cell types or different cell states, though in some contexts it may be more appropriate to think of two cell populations as being of different cell types whereas in other contexts it may be more convenient to think of two cell populations as being of or in different states (though possibly of the same cell type), in which case one would refer to the pure cell signatures of the populations as pure cell state signatures. Where both terms are used together this is simply for clarity rather than to imply a distinction between cell type and cell state.

In another aspect, the invention provides a variety of methods for defining, determining and/or measuring a pure cell type or pure cell state signature. One such method comprises steps of (i) providing a population of cells; (ii) obtaining a gene expression profile for the population of cells across a set of genes, the set comprising at least 10 genes; (iii) repeating the providing and obtaining steps at least once using different populations of cells, thereby generating results for at least two replicates; and (iv) selecting genes whose expression level is consistent among the replicates for use in the pure cell type or pure cell state signature. In various embodiments of the invention the providing and obtaining steps are repeated at least three times, at least four times, at least five times, at least six times, at least seven times, or more.

The foregoing method may be performed using larger numbers of replicates, e.g., three, four, five, six, seven, or more replicates. In certain embodiments of the invention the populations of cells include at least one pure cell population and at least one mixed cell population, e.g., a mixed cell population of known cell type composition. According to certain embodiments of the invention the pure cell type or pure cell state signature comprises expression levels (e.g., RNA or protein levels) of a set of genes in a pure cell population. In various embodiments of the invention the set of genes may comprise at least 10 genes, at least 50 genes, at least 100 genes, at least 500 genes, at least 1000 genes, at least 1500 genes, at least 2000 genes, at least 3000 genes, at least 4000 genes, at least 5000 genes, at least 6000 genes, at least 7000 genes, at least 8000 genes, at least 9000 genes, at least 10000 genes, or more.

In certain embodiments of the invention genes whose expression level is consistent between pure cell populations and/or between substantially identical mixed cell populations are selected for use in defining the pure cell type or pure cell state signature. In certain embodiments of the invention genes whose expression level behaves in a linear fashion across the range of cell type or cell state compositions are selected for use in the pure cell type or pure cell state signature.

The invention also provides various pure cell type or pure cell state signatures for a number of different cell types, obtained according to the inventive methods for obtaining pure cell type signatures. These pure cell type signatures may be used in different embodiments of the invention in order to determine the cell type composition of mixed cell samples. Information identifying the pure cell type signatures may be stored in a database, e.g., on a computer-readable medium. Thus the invention provides a database comprising information identifying at least one pure cell type or pure cell state signature, wherein the database is stored on a computer-readable medium.

In another aspect, the invention provides a computer system for performing the inventive methods for determining the cell type composition of a mixed cell sample. In addition, the invention provides computer-executable process steps stored on a computer-readable medium for performing the inventive methods.

This application refers to various patents, journal articles, and other publications, all of which are incorporated herein by reference. In addition, the following standard reference works are incorporated herein by reference: Current Protocols in Molecular Biology, Current Protocols in Immunology, Current Protocols in Protein Science, and Current Protocols in Cell Biology, John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001.

### Definitions

Cell type or cell state signature: A cell type or cell state signature, as used herein, is the result of a measurement of a set of features, referred to as the signature elements, performed at least once on one or preferably more than one sample(s) consisting of known quantities of cells of that cell type or cell state. A signature element can be, for example, the expression level of an RNA or protein, modification state (e.g., processing state) of an RNA, modification state (e.g., phosphorylation state, glycosylation state, cleavage state, etc.) of a protein, etc. In certain preferred embodiments of the invention the signature elements are measured multiple times using well characterized samples. In certain embodiments of the invention the signature elements are expression levels of mRNA transcripts transcribed from a plurality of genes.

Differential expression: As used herein, a gene exhibits differential expression at the RNA level if its RNA transcript varies in abundance between different samples in a sample set. A gene exhibits differential expression at the protein level, if a polypeptide encoded by the gene varies in abundance between different samples in a sample set. In the context of a cDNA or oligonucleotide microarray experiment, differential expression generally refers to differential expression at the RNA level.

Expression profile: As used herein an expression profile, also referred to as a gene expression profile, is to be given its normal meaning as understood broadly in the art unless otherwise stated. In general, an expression profile may be defined as a dataset that contains information reflecting the absolute or relative expression level of a plurality of genes in a biological sample. The biological sample may range from a single cell (or virus) to a complex population of cells (or viruses) such as that found in a tissue or organ (including both in vivo and in vitro settings such as tissue culture models of biological systems). Generally, an expression profile contains measurements of the expression level of dozens, hundreds, or even thousands of genes. In general, an expression profile reflecting the absolute or relative expression level of an appropriately selected set of genes in a pure population of cells of a particular type constitutes a pure cell type signature for that cell type.

Although the term is most often used in reference to gene expression at the RNA level (e.g., RNA abundance, amount, etc.) as determined, for example, using microarray analysis, it may also or instead reflect expression at the protein level. In general, any measurement technique capable of determining RNA or protein abundance (or abundance of any other biomolecule of interest) may be used to obtain an expression profile. The data may be expressed in any of a number of ways. For example, the data may be expressed in a tabular format, in which entries in the table are numbers that reflect the measured level of expression of a gene in the sample. The data may be transformed in any of a number of ways for ease of analysis and manipulation. Gene expression profiles are frequently displayed in a matrix like format with different colors representing different expression levels, which facilitates a visual understanding of the data.

Although the invention contemplates the use of expression profiles, it is to be understood that other profiles reflective of cell type or cell state may also be used. For example, the invention could make use of “protein modification state profiles” such as phosphorylation state profiles, activity profiles, etc. An activity profile may be defined as a dataset that contains information reflecting the absolute or relative activity of a plurality of biomolecules (e.g., polypeptides) in a biological sample. Any activity may be used, e.g., kinase activity, phosphatase activity, binding activity, inhibitory activity, etc. In general the same activity will be measured for each biomolecule whose activity is included in the activity profile.

Gene: For the purposes of the present invention, the term gene has its meaning as understood in the art. In general, a gene may include gene regulatory sequences (e.g., promoters, enhancers, etc.) and/or intron sequences, 3′ untranslated regions, etc., and coding sequences. It will further be appreciated that definitions of “gene” include references to nucleic acids that do not encode proteins but rather encode functional RNA molecules such as tRNAs, rRNAs, short temporal RNAs (stRNAs), microRNAs (miRNAs), etc. For the purpose of clarity we note that, as used in the present application, the term “gene” generally refers to a portion of a nucleic acid that encodes a protein; the term may optionally encompass regulatory sequences. This definition therefore includes application of the term “gene” to non-protein coding expression units.

Gene product or expression product: As used herein is, a gene product or expression product refers to an RNA transcribed from the gene or a polypeptide encoded by an RNA transcribed from the gene.

Hybridize: The term hybridize, as used herein, refers to the interaction between two complementary nucleic acid sequences. The phrase “hybridizes under high stringency conditions” describes an interaction that is sufficiently stable that it is maintained under art-recognized high stringency conditions.

Isolated: As used herein, isolated means 1) separated from at least some of the components with which it is usually associated in nature; and/or 2) not occurring in nature.

Mixed cell population: The phrase “mixed cell population” refers to any population of cells that includes a cells of a plurality of different cell types and/or cell states. The mixed cell population may occur in vivo or in vitro. According to certain embodiments of the invention a mixed cell population is a cell population present in a tissue or organ (or a portion of a tissue or organ such as a biopsy sample), or in the blood, etc. The term also includes populations obtained by mixing pure cell populations, i.e., populations containing only cells of a single type or state, or by mixing populations of cells that are themselves mixed cell populations. Cell types that may be present in a mixed cell population include, but are not limited to, endothelial cells, muscle cells (e.g., smooth muscle cells, striated muscle cells), fibroblasts, epithelial cells, chondrocytes, osteoclasts, osteoblasts, neurons, glial cells (e.g., astrocytes, oligodendrocytes, microglia), keratinocytes, lymphocytes (e.g., B cells, T cells), monocytes/macrophages, erythrocytes, hepatocytes, pancreatic cells, ovarian cells, testicular cells, glandular cells, endocrine cells (e.g., pancreatic β cells), etc. It will be appreciated that many of the foregoing cell types may be further classified according to any of a number of parameters, e.g., location in the body, etc. For example, endothelial cells exist in vascular structures throughout the body. Endothelial cells may be classified as arterial, venous, or capillary endothelial cells and may also be classified according to the location of the vascular structure. Epithelial cells may be classified as, e.g., respiratory epithelial cells, gastrointestinal epithelial cells, bladder epithelial cells, etc.

According to certain embodiments of the invention the term “mixed cell population” refers to a population of cells that includes cells at a plurality of stages in a differentiation pathway. For example, the population may include chondroblasts and chondrocytes; neuroblasts and neurons; lymphoblasts and lymphocytes, etc. Thus according to certain embodiments of the invention cells at different stages in a developmental pathway (including all varieties of stem cells, progenitor cells, precursor cells, etc.) may be considered distinct cell types or cell states. However, according to certain other embodiments of the invention cells that are at different stages in a single developmental pathway are considered collectively as constituting a single cell type or cell state.

In addition to populations of cells that include a plurality of different cell types, according to certain embodiments of the invention the term “mixed cell population” refers to a population of cells that includes cells of a single type (e.g., cells having the same embryological origin and having followed the same developmental pathway), or of different types, some but not all of which have been exposed to a particular condition or stimulus. Such conditions or stimuli include, but are not limited to, exposure to a growth factor, exposure to a compound such as a toxin or a therapeutic agent, particular pH conditions, temperatures, pressures, concentrations of gases such as oxygen and carbon dioxide, osmotic conditions, radiation, light, etc. Such conditions or stimuli may alter the differentiation pathway followed by the exposed cell. Cells that have been exposed to a particular condition or stimulus may be considered to be of a different state to cells that have not been so exposed. The cell types in a mixed cell population may include cells of a single type wherein all the cells have been exposed to a particular condition or stimulus but only a fraction of the cells display a response thereto.

A “mixed cell population” may also refer to a population that includes cells of a single type or state, wherein some of the cells are normal (healthy) while others are diseased. For example, a mixed cell population may include normal cells of a particular type and also tumor cells arising from the normal cells of that type (e.g., normal breast tissue cells and breast cancer cells; normal cervical epithelial cells and cervical cancer cells, etc.) As another example, a mixed cell population may include uninfected cells of a particular type and also cells of the same type that have been infected by an infectious agent such as a virus, bacterium, parasite, etc. Normal and diseased cells, or uninfected cells and infected cells may be considered as being of different types and/or states or as the same type and/or state for different purposes.

Cell types or cell states can be defined simply by expression profile even in the absence of any otherwise detectable or observable phenotype. Thus any two or more populations of cells that exhibit a different expression profile may be considered as different cell types or cell states.

Purified: As used herein, purified means separated from many other compounds or entities. A compound or entity may be partially purified, substantially purified, or pure, where it is pure when it is removed from substantially all other compounds or entities, i.e., is preferably at least about 90%, more preferably at least about 91%, 92%, 93%, 94%, 95%, 96%, 97%, 98%, 99%, or greater than 99% pure.

Sample: As used herein, a sample may include, but is not limited to, any or all of the following: a virus or viruses, a cell or cells (which may or may not be infected with an infectious agent), a portion of tissue, blood, serum, ascites, urine, saliva, and other body fluids, secretions, or excretions. The cells may be, for example, from blood (e.g., white cells, such as T or B cells) or from tissue derived from solid organs, such as brain, spleen, bone, heart, vascular, lung, kidney, liver, pituitary, endocrine glands, lymph node, dispersed primary cells, tumor cells, or the like. The cells may also be bacterial cells, fungal cells, protozoal cells, etc. Samples may be obtained from a subject by any of a wide variety of methods including biopsy (e.g., fine needle aspiration or tissue biopsy), surgery, collection of body fluid, etc. Samples are not limited to those obtained from a subject but may also be obtained from anywhere in the environment.

The term sample also includes any material derived by processing a sample such as those described above. Derived samples may include nucleic acids or proteins extracted from the sample or obtained by subjecting the sample to techniques such as amplification or reverse transcription of mRNA, in vitro transcription or translation, isolation and/or purification of certain components, etc.

Subject: As used herein, subject refers to any individual including, but not limited to, an individual at risk of or suffering from a disease or clinical condition. The term includes animals, e.g., domesticated animals and wild animals, primates, and humans.

Treating: As used herein, treating includes reversing, alleviating, inhibiting the progress of, preventing, or reducing the likelihood of the disease, disorder, or condition to which such term applies, or one or more symptoms or manifestations of such disease, disorder or condition.

Vector: The term vector is used herein in a biological context to refer to a nucleic acid molecule capable of mediating entry of, e.g., transferring, transporting, etc., another nucleic acid molecule into a cell. The transferred nucleic acid is generally linked to, e.g., inserted into, the vector nucleic acid molecule. A vector may include sequences that direct autonomous replication, or may include sequences sufficient to allow integration into host cell DNA. Useful vectors include, for example, plasmids, cosmids, and viral vectors. Viral vectors include, e.g., replication defective retroviruses, adenoviruses, adeno-associated viruses, and lentiviruses. As will be evident to one of ordinary skill in the art, viral vectors may include various viral components in addition to nucleic acid(s) that mediate entry of the transferred nucleic acid. Preferably, such expression vectors include one or more regulatory sequences operatively linked to the nucleic acid sequence(s) to be expressed.

It is noted that the term vector is also used in its generally understood mathematical sense herein, e.g., to compactly refer to an ordered set of quantities or symbols thereof. Whether vector is used in its biological or mathematical sense will be clear from the context.

### DETAILED DESCRIPTION OF CERTAIN EMBODIMENTS OF THE INVENTION

I. Overview

Most tissues in the body are composed of a complex mixture of different cell types. For example, tumor tissues typically contain a mixture of tumor cells, normal tissue cells, and vascular cells that support tumor growth. Additional cells such as immune system cells may be present as well. In vascular tissues, vessel walls contain smooth muscle cells, endothelial cells, and fibroblasts. Tissue samples, such as biopsy specimens, reflect the complex cell type composition of their source. While studying homogeneous populations of cells such as cell lines removes some of this complexity, in order to understand the molecular mechanisms underlying many biological processes such as cell signaling, it is often necessary to study cell populations containing multiple different cell types and/or cells in multiple different cell states.

The present invention encompasses the inventors' recognition that differences in gene expression profiles between samples containing mixed cell populations reflect not only differences in the various cell types that are present in the samples but also differences in relative cell number and their discovery that it is possible to determine such differences in cell number quantitatively as well as qualitatively using expression profiles. For example, tissue samples obtained from biopsies typically include multiple different cell types, and the proportion of these cell types may vary between samples. In general, the expression profiles of cells of different types over a set of genes will be different, at least with respect to some of the genes. Without a knowledge of the cell type composition of two samples, it is in general not possible to determine whether variations in gene expression between the samples reflects differences in the gene expression pattern of the cells of a single type between the two samples or differences in the relative number of cells of different types between the two samples. While differences in expression profile have been used to identify the presence of particular cell types in tissue samples, such qualitative determinations are of limited utility for various applications such as classifying a disease or evaluating its severity, following the effect of therapy, etc. See, e.g., Perou, C., et al., “Molecular portraits of human breast tumours”, Nature, 406(6797):747-52, 2000, describing use of gene expression profiles to identify the presence of lymphocytes in breast tumor samples.

In general, differences in expression profile between a plurality of samples may reflect differences in cell type composition, differences in expression (on a per cell basis) in cells of the same cell type in the different samples, or both. For example, consider a sample containing a 1:1 ratio of two cell types (50% of each), A and B, in which cell type A expresses gene 1 at a level given by X and cell type B expresses gene 1 at a level of zero, and in which cell type B expresses gene 2 at a level given by X but cell type A expresses gene 2 at a level of zero. It is evident that in such a sample the level of expression of genes 1 and 2 will be equal. Consider a second sample, in which the proportions of the two cell types is unknown and in which the level of expression of gene 1 is twice as high as the level of expression of gene 2(where expression level is measured on a per cell basis). This difference in the expression profiles of the two samples could reflect difference(s) in actual gene expression levels in one or both of the cell types, with the relative proportions of the two cell types being the same in the two samples. For example, if the cells of type B in sample 2 express gene 1 at the same level (X) as the cells of type A (rather than at a level of zero as in sample 1), while the level of expression of gene 2 by cells of type B (X) is the same in the two samples and the level of expression of gene 1 by cells of type A (X) is the same in the two samples and the level of expression of gene 2 by cells of type A is the same in the two samples (zero), then in sample 2 the level of expression of gene 1(X+X=2×, where the first and second terms represent contributions from cell types A and B, respectively) will be twice as high as the level of expression of gene 2(0+X═X, where the first and second terms represent contributions from cell types A and B, respectively). This difference reflects the altered expression of gene 1 in cells of type B in the two samples. Such an alteration could, for example, be an indicator of disease or might be caused by exposure to an agent that stimulates cells of type B to express gene 1. However, the difference in the expression profiles of the two samples could reflect a difference in cell type composition of the two samples with no alteration in the actual levels of gene expression in cells of either type. In this case sample 2 would contain twice as many cells of type A than of type B, i.e., a 2:1 ratio of cell type A to cell type B (˜66.7% cells of type A, ˜33.3% cells of type B), resulting in a 2:1 ratio of expression of genes 1 and 2 in the sample. Alternately, the difference in expression profiles might reflect the presence of a third cell type C in sample 2. For example if cell type C expresses gene 1 at level X and gene 2 at level zero, then a sample containing equal ratios of cell types A, B, and C, would express gene 1 at a level (X+0+X=2×, where the first, second, and third terms represent contributions from cell types A, B, and C respectively) and express gene 2 at a level (0+X+0=X, where the first, second, and third terms represent contributions from cell types A, B, and C respectively). Thus without knowing the cell type composition of sample 2, differences in gene expression profiles of the two samples do not, in general, allow one to unambiguously distinguish differences in gene expression from differences in sample composition. Accordingly, the present invention provides methods and systems for determining the cell type composition of a sample. The invention further provides systems and methods for determining, based on the cell type compositions of two or more samples, whether, and to what extent, differences in measured expression levels of a gene in the two or more samples reflect differences in absolute expression of the gene on a per cell basis or reflect differences in cell type composition of the samples.

II. Using Pure Cell Type Signatures to Determine Cell Type Composition

The present invention provides methods and accompanying computer systems for determining the cell type or cell state composition of a mixed cell population, based on an expression profile, e.g., a gene expression profile, of the mixed cell population. According to the inventive method, pure cell type or cell state specific signatures are defined and measured for each of a plurality of cell types and/or cell states that may be present in a mixed cell population. In general, a pure cell type or cell state specific signature may be thought of as a vector in which each entry reflects the value of a particular signature element, e.g., the level of expression of a particular gene, in a sample consisting only of that cell type or state. For example, if the level of expression of 10 genes is measured, then a pure cell type specific gene expression signature would include an entry for the expression level of each of the 10 genes in a pure population of that cell type. As discussed further below, the invention provides a number of ways to define and measure cell type or cell state specific expression signatures. In particular, cell type or cell state specific gene expression signatures need not be obtained by making measurements on pure populations of cells but can readily be obtained using cell mixtures of known composition. A signature may include entries corresponding to cells of different types, states, or both. For purposes of description the following discussion refers to cell types rather than to both cell types and cell states, but it is to be understood that the pure cell state signatures may be similarly defined and used.

The pure cell type specific signatures for each of a plurality of cell types define the elements of a matrix P, which will be referred to herein as the matrix of pure cell type signatures (or, equivalently, pure cell signatures, pure cell expression signatures, etc.). For example, the columns of P may represent the pure cell type signatures of each of a plurality of cell types, and each row of P may represent the level of expression of a specific gene in each of the different cell types. For example, if it is desired to determine the cell type composition of a tissue sample that may include up to 4 different cell types, A, B, C, and D, then matrix P includes 4 columns, each corresponding to one of the cell types. Each entry in the column corresponding to cell type A reflects the expression level of a different gene in a pure population of cells of cell type A.

In certain embodiments of the invention the cell types whose pure cell type signatures are represented by the columns of P include most or all of the cell types that may be present in a mixed cell population whose composition is to be determined. According to certain embodiments of the invention the cell types whose pure cell type signatures are represented in P include those that together contribute at least 50% of the cells in the mixed cell population. According to certain embodiments of the invention the cell types whose pure cell type signatures are represented in P include those that together contribute at least 75% of the cells in the mixed cell population. According to certain embodiments of the invention the cell types whose pure cell type signatures are represented in P include those that together contribute at least 85% of the cells in the mixed cell population. According to certain embodiments of the invention the cell types whose pure cell type signatures are represented in P include those that together contribute at least 90% of the cells in the mixed cell population. According to certain embodiments of the invention the cell types whose pure cell type signatures are represented in P include those that together contribute at least 95% of the cells in the mixed cell population. According to certain embodiments of the invention the cell types whose pure cell type signatures are represented in P include those that together contribute 99% or more of the cells in the mixed cell population.

In general, the matrix P may be represented as shown below.

$$r rows {( \begin{matrix}{a}_{11} & {a}_{12} & {a}_{13} & … & {a}_{1 c} \\ {a}_{21} & {a}_{22} & & & ⋮ \\ {a}_{31} & & & & \\ ⋮ & & & & \\ {a}_{r1} & … & & & {a}_{rc}\end{matrix} )}^{c columns}$$

P contains r rows and c columns. Thus the data in the matrix reflects the level of expression of each of r genes in each of c different pure populations of cell types. Each entry aᵢⱼ represents the expression level of gene i in a pure population of cells of type j. In certain embodiments of the invention the entries in each column of P represent the expression level of the various genes in a unit quantity of the relevant cell type. The unit quantity may be given in terms of number of cells of the relevant cell type, amount of total or poly A⁺ RNA used to measure the expression levels for the relevant cell type, or any other suitable parameter. For example, a column may represent the expression profile that would result from measuring expression in a pure population of 1 million cells of the type corresponding to that column (though the expression profile need not result from measurements made on a pure population). Alternately, a column may represent the expression profile that would result from measuring expression in 1 μg of total RNA isolated from cells of the type corresponding to that column. In certain embodiments of the invention the unit quantities are the same for each column (i.e., for cells of each type). For purposes of description it will be generally be assumed herein that the unit quantities used to obtain the pure cell type signatures of the various cell types are the same, i.e., that P is a matrix of pure unit cell type signatures.

If the unit quantities are not the same, the entries in P should be standardized to account for that fact. For example, if a quantity of 1 μg of RNA was used to measure expression for two cell types and a quantity of 10 μg of RNA was used to measure expression for a third cell type, the expression levels for the third type should be multiplied by 0.1 so that the same unit quantity is used for all entries in the matrix of pure cell type signatures. This may be accomplished by multiplying the matrix P by a suitable matrix to obtain a standardized matrix PST, which is then used instead of P. Example 3 illustrates the standardization process in the context of a particular set of pure cell type signatures. If the unit quantities are not cell numbers, e.g., if they are amounts of total or poly A⁺ RNA used to measure the expression levels for the relevant cell type, then an entry for a cell type X in the vector q will represent the amount of RNA from cells of cell type X present in the sample. It may be desirable to convert the amounts of RNA into absolute cell numbers. In general, in order to do so it is necessary to know the approximate amount of RNA per cell for each cell type, or preferably, the amount of RNA per cell type that is extracted using whatever technique is used to isolate RNA from that cell type in the practice of the invention. This measurement may be made using standard RNA quantification techniques, e.g., optical density, or any other appropriate technique. The amount of RNA per cell serves as a conversion factor that may be used to convert the entries in vector q into absolute cell numbers by dividing the entry for a given cell type in q by the amount of RNA per cell in cells of that cell type, or equivalently, multiplying the entry by the reciprocal of that quantity. For example, the inventors determined that endothelial cells contain ˜40 pg RNA/cell (i.e., harvesting RNA from ˜250,000 EC yielded 10 ug RNA). Similarly, smooth muscle cells contain ˜16 pg/cell (i.e., harvesting RNA from 625,000 SMC yielded 10 ug RNA), and fibroblasts contain ˜10 pg/cell (i.e., harvesting RNA from ˜1,000,000 fibroblasts yielded 10 ug RNA).

If q is a vector whose elements represent the number of each cell type present in a mixed population of cells, then according to the invention it is desired to determine the values for the elements of q by measuring the expression profile m of the mixed population. Note that consistent with the above equation, q is a column vector in which the number of rows equals the number of columns of P, and the ith element in q represents the number of cells (in the mixed cell population) of the type whose pure cell expression signature is given by the ith column of P. The inventors have recognized that, assuming linearity of expression, then the following matrix equation holds, where Pq is the product of matrix P and vector q:
Pq=m (Eq. 1)

Since m is measurable, equation 1 can be solved to obtain values for the entries in q. These values are the number of cells of each cell type present in the sample, expressed in terms of the unit quantity of that cell type (i.e., the unit quantity that was used in determining the coefficients in the matrix P of pure cell signatures). In general, equation 1 may not be directly solvable. Instead, according to certain preferred embodiments of the invention an approximate solution is computed. Generally, in preferred embodiments of the invention a least squares solution is computed. Explicitly, to compute an estimate of the vector of quantities q representing a sample with expression profile m, the following equation is used:
q*=LSQ[Pq=m]=(P^{T}P)⁻¹P^{T}m (Eq. 2)

In the above equation q* represents the least squares solution of equation 1, P^{T} represents the transpose of matrix P, and (P^{T}P)⁻¹ represents the inverse of matrix (P^{T}P). It is noted that the expression (P^{T}P)⁻¹P^{T} is the pseudoinverse of matrix P. (According to the notation used herein, when symbols representing two matrices, vectors, etc., are presented consecutively, without intervening spaces, it is to be understood that the matrices, vectors, etc., are to be multiplied, unless otherwise stated.) If m*=Pq*, and if corresponding entries in m and m* (i.e., entries that reflect the number of cells of a particular cell type) are designated by identical subscripts, e.g., m=[m₁, m₂, m₃, . . . mₙ] and m*=[m₁*, m₂*, m₃*, . . . , mₙ*] (but note that m and m* are column vectors) then the least squares solution minimizes the sum of the squares of the errors, i.e., the least squares solution minimizes (m₁−m₁*)²+(m₂−m_{2*})²+(m₃−m₃*)²+ . . . +(mₙ−mₙ*)². In other words, the least squares solution, q*, minimizes norm(m−Pq).

Approximate solutions to equation 2(such as least squares solutions) may readily be computed using algorithms that are well known in the art and can be performed using standard mathematical software such as Matlab™ (The MathWorks, Inc., 3 Apple Hill Drive, Natick, Mass. 01760-2098, Mathematica™ (Wolfram Research, Inc., 100 Trade Center Drive, Champaign, Ill. 61820-7237), or similar programs capable of performing matrix algebra. General discussions of linear algebra and methods for computing solutions to the equations presented herein may be found in, e.g., Golub, G. H. and Van Laon, C. F. (1989) Matrix Computations, Baltimore Md.: Johns Hopkins University Press. The Matlab™ software package has standard functions lsqr( ) and lsqnormeg( ) that implement the least squares algorithm. The latter function finds a solution with nonnegative coefficients, which is appropriate for the applications described herein. Example 3 describes the use of Matlab instructions to solve equation 2 in the context of particular pure cell type specific signatures.

In certain embodiments of the invention the user selects a matrix of pure cell type signatures P (i.e., coefficients for P) from a set of predetermined matrices corresponding to different cell types. For example, if the sample contains. EC, SMC, and FC, the user may select a matrix of pure cell type signatures that includes cell type signatures for those cell types. Alternately, in certain embodiments of the invention the user enters the cell types expected to be present in the sample, and the program selects an appropriate matrix. The set of predetermined matrices may be stored in a database on the computer system. In certain embodiments of the invention the user may enter coefficients for a pure cell type signature to be used in determining the cell type composition of a sample.

One aspect of the invention is the inventors' discovery that when appropriate pure cell signatures are used, the assumption that expression behaves in a linear fashion is sufficiently valid for mixed populations of cells so that equation 1 may be used to solve for the cell numbers in mixed cell populations. The general concept of linearity is well known in the art. In the context of the present invention, the assumption of linearity may be described as follows: Let E_{Y}(X) represent the expression of a gene Y in a unit number of cells of type X. Then for two cell types, A and B, linearity of expression implies that E_{Y}(c₁A+c₂B)=c₁E_{Y}(A)+c₂E_{Y}(B), where c₁ and c₂ are arbitrary constants, generally greater than or equal to 0. The preceding equation may readily be generalized to include any number of different cell types. It is to be understood that linearity need not apply to every gene in a pure cell type signature. In addition, expression need not be perfectly linear. Approximate linearity is sufficient.

The degree to which expression of any particular gene or set of genes is linear across different samples, different experimental conditions, etc., may be determined experimentally, e.g., by (i) measuring the expression levels for the genes in the different samples, under the different experimental conditions; (ii) counting the number of cells of each different cell type; and (iii) calculating the expression level of the gene or set of genes on a per cell basis for each sample and/or each experimental condition. For genes whose expression behaves in a linear fashion the per cell expression levels should be approximately the same in the different samples or under the different experimental conditions.

As described in Examples 3, 4, and 5, the inventors have shown that pure cell type signatures obtained for smooth muscle cells, endothelial cells, and fibroblasts from blood vessel origin can be used to determine the composition of samples containing mixtures of these cell types. Samples with a wide range of compositions were tested. This finding confirms the assumption of linearity and demonstrates the validity of the approach.

III. Detecting Cell State or Cell Stimulation

The discussion above has described the use of the inventive methods to determine the cell type or cell state composition of mixed cell samples of unknown composition. In general, the cell states may be any biochemical or physiological states including, but not limited to, (1) normal and diseased states; (2) states of exposure to different conditions or environments, e.g., different pH or temperature; (3) treated and untreated states, which may include exposure to a variety of different treatments, doses, etc.; (4) developmental states, e.g., cells at different stages of a differentiation pathway; (5) wild type and mutant states; (6) infected and non-infected states; (7) cells in different stages of the cell cycle, etc. In general, the methods may be employed to determine the number of or detect the presence of cells that have been subjected to stimulation or to any condition that induces a change in cell state that is reflected in an alteration in gene expression pattern (which may or may not be reversible). As is well known in the art, cells may alter their gene expression pattern in response to a wide variety of environmental conditions and stimuli. By “stimulus” is meant any agent capable of eliciting a change in the expression level of at least one signature element (e.g., the expression level of a gene), or any chemical, physical, or biological condition capable of eliciting such a change. The change may be an increase or a decrease in gene expression. In general, many stimuli act via signaling pathways that lead to the activation or inhibition of transcription factors, which then act to alter RNA transcription.

Representative examples of chemical stimuli include growth factors, cytokines, hormones, and numerous small molecules used for therapeutic purposes. Representative examples of stimuli that may be classified s biological stimuli include, e.g., cell-cell contacts, cell contact with extracellular matrix, entry of an infectious agent, etc. Physical stimuli include changes in temperature or pressure (e.g., changes in pressure in blood vessels occurring during the cardiac cycle or in tissue culture), changes in the ionic composition or concentration of the extracellular environment, etc. Note that such classifications are merely for the sake of convenience and are not absolute. In many situations a multiplicity of stimulating factors may be identified. For example, when an artery is subjected to a procedure such as percutaneous transluminal balloon angioplasty (PTCA), cells in the arterial wall are exposed to numerous stimuli including pressure from the balloon and numerous compounds released from cells that are damaged by the procedure.

Stimulated and unstimulated cells of a single type may be thought of as two distinct cell types, or two distinct cell states, in which case the methods described above are directly applicable. According to the invention pure cell type signatures are obtained for cells in their unstimulated and stimulated conditions, and these pure cell type signatures constitute columns in P, the matrix of pure cell type signatures. In general, if it is desired to determine the cell type composition and the numbers of stimulated and unstimulated cells in a mixed cell composition, a matrix PN of pure cell signatures for each of the various cell types in their unstimulated (normal) state is obtained. A similar matrix Ps of pure cell signatures for each of the various cell types in its stimulated state is obtained. These matrices may be concatenated to form the larger matrix [PNPS], which corresponds to matrix P above. (Note that here the juxtaposition of the PN and PS does not indicate multiplication but rather concatenation.) Thus
[P_{N}P_{S}]q=m (Eq. 4)
where m is a measured gene expression profile for the mixed cell sample and q is a vector of quantities representing the number of each cell type, with separate entries for stimulated and unstimulated cells of each type, in the sample. Pure cell signatures for stimulated and unstimulated cells may be obtained from pure cell populations, which may be exposed to a stimulus of interest in vitro or in vivo. For example, a pure population of cells may be maintained in tissue culture and split into two portions, one of which is exposed to the stimulus (e.g., addition of a growth factor to the medium). Both portions are subsequently harvested, and pure cell type signatures obtained for each portion.

It is noted that gene expression patterns may change over time in response to a stimulus. As just one example, it is well known that mitogenic stimuli lead to the rapid activation of a subset of genes (early genes), followed later by increased transcription of additional genes important in the cell division cycle. The expression of any particular gene may eventually reach a new steady state or may return to its original expression level. Thus it may be desirable to obtain pure cell type signatures at a range of time points following application of the stimulus. Analogous to the methods described above for obtaining pure cell type signatures from mixed cell samples of known composition, pure cell type signatures for stimulated and unstimulated cells may be obtained using measurements made on mixed populations of known compositions, i.e., populations in which the proportion of cells of different types and the proportion of stimulated and unstimulated cells of each type are known.

IV. Determining Contribution of Changes in Absolute Expression Levels versus Differences in Cell Type Composition to Measured Differences in Expression Levels

The methods of the invention are useful in determining whether a difference in gene expression profile between two or more samples results from changes in gene expression on a per cell basis (referred to as “actual changes” in gene expression) or is due to differences in cell type composition of the samples. If it is found that two samples do differ in cell type composition, it may be desirable to determine whether such differences are responsible for any detected differences in gene expression profile and, if so, what contribution they make. For example, suppose that a first sample containing cells of three different types is determined to have a cell type composition ratio of 1:1:8, and a second sample containing cells of these types is determined to have a cell type composition ratio of 1.5:1:7.5. In general, the gene expression profiles cannot be directly compared to infer gene expression levels in cells in the samples since it would not be possible to determine whether differences resulted from actual changes in gene expression or were a consequence of the different proportions of cells.

Accordingly, the invention provides methods and systems for determining, based on the cell type compositions of two or more samples, whether, and to what extent, differences in measured expression levels of a gene in the two or more samples reflect differences in absolute expression of the gene on a per cell basis or reflect differences in cell type composition of the samples. In particular, the invention provides a method for determining whether a difference in measured expression level of a gene in first and second samples reflects a difference in absolute expression of the gene on a per cell basis or reflects a difference in cell type composition of the samples comprising steps of: (i) providing or determining the cell type composition of the first sample; (ii) providing or determining the cell type composition of the second sample; and (iii) determining, based on the cell type compositions of the two samples, whether a difference in expression level of the gene between the two samples reflects a difference in absolute expression on a per cell basis or a difference in cell type composition of the two samples. The invention may further include steps of measuring the expression level of the gene in one or both samples. According to certain embodiments of the invention the method is applied to an experimental sample which is compared with a control or reference sample with a known cell type composition and expression level. The method may be applied to multiple samples, e.g., by considering the multiple samples pairwise.

According to certain embodiments of the invention the determining step (i.e., the third step) comprises (i) comparing the cell type composition of the first and second samples; and (ii) if the cell type composition of the first and second samples are substantially the same, inferring that any differences in expression of the gene are actual changes in expression. According to certain embodiments of the invention the determining step comprises (i) comparing the cell type composition of the first and second samples; and (ii) if the cell type composition of the first and second samples are not substantially the same, inferring that any differences in expression of the gene arise at least in part as a result of differences in cell type composition of the samples. The determining step may also comprise correcting the measured expression level of the gene in the second sample to reflect the expression level that would have resulted if the two samples had contained the same relative numbers of cells of each type, i.e., if the two samples had the same cell type composition.

It will be evident that two samples are unlikely to have identical cell type compositions. The extent to which two slightly different cell type compositions can be considered substantially the same or identical may be defined in various ways depending on the particular application and purpose of the analysis and the accuracy required. For example, two samples may be considered to have substantially the same cell type composition if the proportion of each cell type in the second sample is within ±1%, ±5%, ±10%, ±15%, ±25%, or ±50% of the proportion of that cell type in the first sample. Any other value may be selected, with lower numbers being preferred. Alternately, a least squares metric may be used. The percentage difference between any two values A and B may be determined by computing the absolute value of either (A−B)/A or (A−B)/B and multiplying the resulting number by 100. According to various embodiments of the invention the cell type composition of two samples is substantially the same if the percentage of every cell type represented in the determined cell type composition is substantially the same in both samples. According to other embodiments of the invention the percentage of one or more of the cell types represented in the determined cell type composition may not be substantially the same in both samples, provided that the percentage of at least one of the cell types is substantially the same.

The availability of pure cell type signatures allows the gene expression profile for the second sample to be transformed into a gene expression profile that would have been obtained if the second sample had exactly the same cell type composition as the first sample. The first sample may be, for example, a reference sample. By correcting gene expression profiles to reflect results that would have been obtained if a set of samples contained a standard cell type composition, differences in actual gene expression can be detected and compared. In addition, the availability of pure cell type signatures makes it possible to completely remove the contribution of one or more cell types to a gene expression profile, thus allowing the researcher or clinician to focus on analysis of the remaining cell types. These methods are of particular use for a wide variety of research and diagnostic applications.

V. Defining Pure Cell Type or Cell State Signatures

The invention provides a variety of ways to define a pure cell type signature for any given cell type, any of which may be used in the practice of the methods described herein. By “defining a pure cell type signature” is meant selecting the set of signature elements whose values will be included in the pure cell type signature for a particular cell type. For example, the signature elements may be expression levels of genes that will be included in the pure cell type signature for a particular cell type. Thus in certain embodiments of the invention a pure cell type signature is a dataset that includes the level of expression of a plurality of genes for a pure cell population of that cell type (though as mentioned above a pure cell signature may be derived from measurements made on mixed cell populations of known composition). Different pure cell type signatures will result from different selections of genes whose expression level is to be included in the pure cell type signature. Thus determining a pure cell type signature includes two distinct steps: (1) selecting appropriate genes (i.e., defining the signature); and (2) measuring the expression level of the selected genes in a pure cell population (or deriving the expression level from mixed cell populations of known composition). In various embodiments of the invention these steps can be performed in either order.

According to certain embodiments of the invention a pure cell type signature is obtained by measuring the expression level of a plurality of genes that are selected without reference to the characteristics of the particular cell type, e.g., in a random or semi-random fashion (referred to herein as an unbiased pure cell type signature). Such genes may be representative of overall gene expression in an organism or tissue or may have been selected in a particular manner unrelated to the properties of the cell type. In general, any set of genes whose selection was not intentionally biased in favor of including or excluding genes that are either overexpressed or underexpressed in the cell type of interest is suitable for determination of a pure cell type specific signature. According to certain embodiments of the invention a pure cell type signature is obtained by measuring the expression level of a plurality of genes that are selected with reference to the characteristics of the particular cell type. For example, the genes may be selected to include genes known (e.g., from the literature or from earlier experiments) to be overexpressed or underexpressed in that cell type. Such genes can be identified using any of a variety of techniques, e.g., subtractive hybridization. When genes are selected based upon their expression level the signature may be referred to as an expression biased pure cell type signature.

According to certain embodiments of the invention a pure cell type signature for a first cell type is obtained by measuring the expression level of a plurality of genes that are selected with reference to the characteristics of a second cell type that is likely to be present in the tissue or organ in which the first cell type is found within the body. The genes may be selected to include genes known to be overexpressed or underexpressed in the second cell type relative to the first cell type or relative to any other cell type. For example, vessel walls contain endothelial cells, smooth muscle cells, and fibroblasts in varying proportions. A pure cell signature for fibroblasts may be obtained by measuring the expression level of a plurality of genes that are selected because they are overexpressed in endothelial cells.

According to certain embodiments of the invention a pure cell type signature is obtained by measuring the expression level of a plurality of genes that are selected with reference to the characteristics of a tissue or organ in which the cell type is typically found within the body. The genes may be selected to include genes known to be overexpressed or underexpressed in the tissue or organ relative to other tissues or organs or relative to a reference cell type, etc. For example, a pure cell type signature for fibroblasts may be obtained by measuring the expression of a set of genes known to be overexpressed in vascular tissue since fibroblasts are typically found in vascular tissue as well as in other tissue types.

According to certain embodiments of the invention a pure cell type signature is obtained by measuring the expression level of a plurality of genes that are selected with reference to the characteristics of the cell type for which the pure cell type signature is to be obtained. The genes may be selected to include genes known to be overexpressed or underexpressed in the cell type relative to one or more other cell types or relative to a reference cell type, etc. For example, a pure cell type signature for fibroblasts may be obtained by measuring the expression of a set of genes known to be overexpressed or underexpressed in fibroblasts. As another example, a pure cell type signature may be obtained by measuring the expression of a set of genes whose expression is known to increase or decrease in cells of a particular type in response to exposure to a condition or stimulus. Pure cell type signatures selected with reference to the characteristics of the cell type for which the pure cell type signature is to be obtained may be particularly useful where it is desired to obtain a qualitative determination of whether a particular cell type is present or absent in a sample, which may be done instead of or in conjunction with performing a quantitative determination of cell type composition. For example, such a step may be performed prior to obtaining a quantitative determination and may be used to determine which particular pure cell type signatures should be used for the quantitative determination of cell type composition. For example, if it is determined that the sample contains lymphocytes, it may be desirable to include a pure cell type signature for lymphocytes in the matrix of pure cell type signatures, whereas if it is determined that the sample does not contain lymphocytes it may be preferable not to include a pure cell type signature for lymphocytes in the matrix of pure cell type signatures.

According to certain embodiments of the invention genes whose expression level exhibits a relatively low degree of variability when measured in samples that represent multiple replicates of substantially identical cell type composition and experimental conditions are selected for use in defining a pure cell type signature. Such genes may be referred to as consistent genes and their expression level may be considered to exhibit consistency. By “substantially identical cell type composition” is meant that the cell type composition, with respect to one or more cell types, varies by less than a preselected percentage, e.g., 1%, 5%, 10%, 25%, etc., depending on the particular embodiment of the invention. Substantially identical experimental conditions are intended to include those conditions under the deliberate control of the experimenter, e.g., temperature, media composition, etc. It will be appreciated that if cell type composition and experimental conditions were truly identical then variations in expression between samples would be minimal. However it is impossible to accurately control numerous variables that may influence expression levels. By identifying those genes that exhibit consistency, one selects the genes that are least affected by variations in experimental conditions that are outside the control of the experimenter.

According to certain embodiments of the invention genes whose expression level varies by less than 20% when measured in multiple samples with substantially identical composition and experimental conditions are included. By “varies by less than X %” is meant that within a set of replicates all values lie within X % of the mean value. According to certain embodiments of the invention genes whose expression level varies by less than 10% when measured in multiple samples with substantially identical composition and experimental conditions are included. According to certain embodiments of the invention genes whose expression level varies by less than 5% when measured in multiple samples with substantially identical composition and experimental conditions are included. According to certain embodiments of the invention genes whose expression level varies by less than 2% or less than 1% when measured in multiple samples with substantially identical composition and experimental conditions are included.

For example, if gene expression is measured using microarrays, genes with variation in log ratio in replicate experiments less than 0.1 if the background-subtracted signal in the sample channel for the genes is less than 1000 are selected for use in defining the pure cell type signature of a cell type. By “log ratio” is meant log (signal from test sample/signal from reference sample), e.g., (Cy5 signal/Cy3 signal) where the reference RNA is labeled with Cy3 and the test sample is labeled with Cy5. According to certain embodiments of the invention genes with variation in log ratio less than 0.2 in replicate experiments if the background-subtracted signal in the sample channel for the genes is more than 1000 but less than 20000 are selected for use in defining the pure cell type signature of a cell type. According to certain embodiments of the invention genes with variation in log ratio less than 0.3 in replicate experiments if the background-subtracted signal in the sample channel for the genes is more than 20000 are selected for use in defining the pure cell type signature of a cell type. Any number of replicates may be measured, e.g., between 2 and 10 replicates, or more. It is assumed that replicates are performed using samples of substantially identical cellular composition and under substantially identical experimental conditions. In general, the larger the number of replicates, the more strongly one can conclude that the gene exhibits consistent expression. According to certain embodiments of the invention a number of replicates sufficiently large to afford statistical significance that the expression level falls within a specified confidence interval is selected. For example, the number of replicates may be selected to provide a p value of <0.1, <0.05, etc.

It is noted that although expression levels may be represented as log ratios, the entries in P should be either absolute numbers (e.g., signal from red channel) or ratios (e.g., signal from red channel divided by signal from green channel) but should not be log ratios. The term “expression level” as used herein therefore generally refers either to absolute numbers or to ratios rather than log ratios. It is to be understood that the foregoing description is for representative purposes only. One of ordinary skill in the art will be able to select appropriate parameters by which to identify genes whose expression is consistent across multiple samples, depending, for example, on the particular methods and equipment used to measure expression.

According to another approach, rather than selecting genes whose expression level among replicates varies by less than a specified amount, one simply selects genes whose expression is most consistent, regardless of the specific method used to evaluate consistency and regardless of the actual level of consistency. For example, according to certain embodiments of the invention one selects 1% of the total number of genes, 2%, 5%, 10%, etc. Any other percentage, either smaller than 1% or larger than 10% can also be used. The percentage may be selected so as to include a predetermined number of genes and may thus vary depending on the total number of genes. According to certain embodiments of the invention the total number of genes is considered to be the total number of genes present in or identified in the genome of a cell type of interest (i.e., the total number of genes present in or identified in the genome of an organism from which the cell type originates). According to certain embodiments of the invention the total number of genes is considered to be the number of genes whose expression is measured to determine an expression profile, e.g., the number of genes (or clones) represented on a microarray in the case of a microarray measurement. According to certain embodiments of the invention the total number of genes is considered to be the number of entries in the vector m as defined above. In general, any appropriate method of selecting genes that exhibit consistent expression levels can be used, and one of ordinary skill in the art will be able to select an appropriate method having regard for the experimental conditions under which the genes are selected.

According to certain embodiments of the invention genes selected for use in the pure cell type signature exhibit consistency when tested in multiple samples having a range of cell type proportions. For example, it may be desirable to include genes whose expression level exhibits consistency when measured in multiple samples of substantially identical cell type composition (i.e., multiple replicates) in which the cell type is present as a relatively small percentage of the total cell number (e.g., less than 20%, less than 10%, or less than 5% of the total cell number) and also exhibits consistency in samples of substantially identical cell type composition (i.e., multiple replicates) in which the cell type is present as an intermediate or relatively large percentage of the total cells (e.g., greater than 30%, greater than 40%, greater than 50%, greater than 60%, greater than 70%, greater than 80%, or greater than 90% of the total cell number). In any of the foregoing embodiments described herein the number of repetitions used to determine whether expression is consistent can be, e.g., any number between 2 and 10, or more.

According to certain embodiments of the invention genes for defining a pure cell type expression profile are genes whose expression level varies significantly between different cell types whose presence or relative number in a sample is to be determined, i.e., genes that exhibit significant differential expression. For example, and without intending to be limiting, according to various embodiments of the invention genes whose expression level varies by at least a factor of 1.5, at least a factor 2, at least a factor of 3, at least a factor of 4, at least a factor of 5, at least a factor of 10, etc., between two or more cell types or between any two cell types may be selected. By “at least a factor of X” is meant that the expression level of a gene Y in cell type 1 is at least X times the expression level of the gene in cell type 2. Significant differential expression may be defined in a number of ways, e.g., in terms of percentage overexpression in one cell type relative to another cell type or relative to the average expression level in one or more cell types. In addition, differential expression may be expressed in terms of differences between the log ratios of expression in different cell types relative to a common reference sample. For example, and without intending to be limiting, according to certain embodiments of the invention genes whose expression level has at least a difference in log ratio of at least 0.125, at least 0.25, at least 0.3, at least 0.4, at least 0.5, at least 0.6, at least 0.7, at least 0.8, at least a 0.9, at least 1.0, etc., between two or more cell types or between any two cell types may be selected.

Two or more of the above criteria may be used to select genes for use in a pure cell type signature. For example, an initial set of genes may be selected according to an expression biased approach, e.g., genes that are overexpressed in a particular tissue type. Then a subset of these genes that exhibit consistency may be selected for use in the pure cell type signature for cells found in the tissue. The number of genes included in a pure cell type signature defined according to any of the above criteria may vary. According to certain embodiments of the invention the set of genes includes at least 10 genes, at least 20 genes, at least 50 genes, at least 100 genes, between 100 and 500 genes, between 500 and 1000 genes, between 1000 and 2000 genes, between 2000 and 3000 genes, between 3000 and 4000 genes, between 4000 and 5000 genes, or more than 5000 genes.

In general, a primary determinant of whether a set of genes is suitable for use in defining a pure cell type signature for a particular cell type is whether the expression level of the set of genes satisfies the assumption of linearity discussed above, preferably over a range of sample characteristics typical of those for which the cell type composition is to be determined. The above discussion has merely identified several possible approaches to the selection of an appropriate set of genes for use in defining a pure cell type signature. However, any set of genes may readily be tested to determine whether it satisfies the assumption of linearity. This may be done, e.g., by obtaining gene expression levels for the genes using samples of known composition, using these as entries in the matrix P as described above, computing the solution q* for the equation Pq=m using these entries and determining whether q* yields the known cell type composition. If q* yields accurate values over samples with a range of different cell type compositions, then the set of genes is appropriate for defining pure cell type signatures for cells in the compositions. Examples 2, 3, and 4 provide further details. As will be evident to one of ordinary skill in the art, whether the expression of any particular gene satisfies the assumption of linearity may vary depending on the technology employed to measure expression. Thus results obtained using one technology may not necessarily be valid when a different technology or measurement technique is employed. Thus in general selection of an appropriate set of genes for use in a pure cell type signature, and also measurement of the pure cell type signature, should be done using the same measurement technology or technique (or a sufficiently similar measurement technology or technique so that results will be approximately the same) as that which will be employed to determine the cell type composition of a sample or to practice the other methods of the invention. Alternatively, where systematic differences in results obtained using different measurement technologies or techniques exist, corrections can be made to account for such differences.

VI. Determining Values for Pure Cell Type Signatures.

A. Obtaining Pure Cell Type Signatures Using Pure Cell Populations

Given a set of genes whose expression levels constitute a pure cell type signature, one way to determine the coefficients of P for a particular cell type (i.e., the pure cell type signature for that cell type) is to measure the level of gene expression for the set of genes in a pure population of cells of that type. Such measurements may conveniently be performed using microarrays to obtain gene expression profiles, as described in more detail in the following section. Alternately, any of a wide variety of other methods may be used as also described below. Pure cell populations may be obtained in any of a number of ways. According to certain embodiments of the invention a cell line is used as a source of a pure population of cells. Numerous cell lines that originate from cells of many different cell types are known in the art. In general, a cell line may be considered to have the same cell type as the cell or cells from which it originated. In many cases the gene expression profiles of a cell line corresponds closely with a gene expression profile obtained from primary cells of the same type (i.e., cells obtained from an organism or tissue source that not been passaged (split) in tissue culture). Numerous well characterized cell lines are available, e.g., from the American Type Culture Collection (see Web site having the URL www.attc.org) and from commercial suppliers.

In general, cell lines differ from their counterparts in the body and/or from primary cells in that they are immortal, i.e., they do not senesce. This difference may be due to or may contribute to differences in gene expression between cell lines and primary cells and/or their counterparts in the body. In addition, mutations occur as cells are maintained, and a process of selection takes place such that the phenotypic characteristics of the cells change over time. These phenotypic changes may reflect changes in gene expression patterns. Therefore, although certain cell lines may be an appropriate source of cells for some cell types, according to certain embodiments of the invention it is preferable to avoid using cell lines but rather to use primary cells or cells that have undergone only a small number of passages and/or cell division cycles in culture. For example, according to certain embodiments of the invention cells that have undergone twenty or less passages and/or cell division cycles in culture are used. According to certain embodiments of the invention cells that have undergone ten or less passages or cell division cycles in culture are used. According to certain embodiments of the invention cells that have undergone five or less passages or cell division cycles in culture are used. According to certain embodiments of the invention cells that have undergone two or less passages or cell division cycles in culture are used. According to certain embodiments of the invention cells that have not been maintained in tissue culture or have been maintained for less than 24 hours are used (i.e., cells isolated directly from an organism or tissue sample).

Methods for obtaining pure populations of cells from tissue samples are well known in the art for a wide variety of cell types. Cells can be separated based on their phenotypic features, growth characteristics (e.g., requirement for a substrate, requirements for particular components in the culture medium, requirements for particular growth conditions, etc.), or based on their expression of particular markers. For example, FACS using fluorescent antibodies that bind to specific cellular markers characteristic of a particular cell type can conveniently be used to separate cells of that type from cells of other types. Pure populations of cells of low passage number may be obtained from various commercial suppliers (e.g., Clonetics, Inc.). Note that a “pure” population of cells need not be 100% pure, i.e., it need not consist entirely of cells of a single cell type. However, preferably a pure population of cells has a high degree of purity, e.g., at least 90%, at least 95%, at least 98%, at least 99% or between 99% and 100%.

The number of cells in a pure cell population to be used in obtaining a pure cell type signature may vary and an appropriate number may depend upon the particular experimental techniques used to determine the gene expression levels. One of ordinary skill in the art will be able to determine an appropriate number. For example, if a standard microarray analysis is performed, a number of cells sufficient to provide approximately 10 jig of total RNA may be used. Thus the appropriate number of cells will vary depending on the average RNA content per cell. The inventors have typically used approximately 250,000-300,000 endothelial cells, 450,000-600,000 smooth muscle cells, and 350,000-500,000 fibroblasts, for cell mixing experiments. However, these numbers are only intended to be representative of suitable ranges of cell numbers. In certain embodiments of the invention much smaller numbers of cells are used, possibly as few as a single cell. The invention contemplates the use of amplification techniques, preferably linear amplification techniques, to obtain sufficient RNA for analysis in appropriate situations.

B. Obtaining Pure Cell Type Signatures Using Mixed Cell Samples of Known Composition

Although pure cell type signatures may be conveniently obtained by measuring gene expression in pure cell populations, according to certain embodiments of the invention such measurements may be performed on samples of known composition rather than on pure samples. According to certain embodiments of the invention samples of known composition are obtained by mixing pure cell populations in known proportions. For example, it may be desirable to obtain pure cell type signatures under conditions in which cells can interact with one another, or it may be desirable to obtain cell type signatures using mixed cell samples isolated from an organism or tissue since gene expression patterns in such situations may differ from those obtained when cells are maintained in tissue culture.

Pure cell populations obtained as described above can be mixed in known proportions and cultured together for a period of time (e.g., to allow cell interaction) prior to measuring the gene expression levels. If the culture period is longer than the cell cycle time of any of the cells in the mixture, cell numbers must be adjusted accordingly. Alternately, a tissue sample (e.g., a section of an artery) can be harvested. The cell type composition of the sample can be determined using any of a variety of techniques (e.g., visual observation under a microscope, FACS using cell type specific antibodies, etc.). To obtain mixed cell compositions having a variety of different cell ratios, cells of different types may be isolated from the tissue sample (e.g., using visual observation and microdissection, laser capture microdissection, and/or FACS using cell type specific antibodies) and then mixed together in known proportions.

Given measurements performed on samples of known composition, the pure cell type signatures may be derived as follows: Let G be a matrix whose columns represent the known compositions of the samples in which gene expression is measured. The number of entries in each column is equal to the number of cell types in the samples. Thus if gene expression levels are measured in five samples, each of which contains up to three different cell types (cell types A, B, and C), G would contain five columns, each containing three entries, one of which corresponds to each cell type. For example, the first entry in each column might represent the number of cells of type A in the sample corresponding to that column; the second entry in each column might represent the number of cells of type B in the sample corresponding to that column, etc. In general, the ith entry in each column represents the number of cells of type i in the sample corresponding to that column. The numbers need not be, and in general will not be absolute cell numbers but will instead be normalized to account for the fact that different samples may contain different total cell numbers. Thus generally the numbers will be a percent, a fraction, etc., reflecting the contribution that each cell type makes to the total cell number in the sample. For example, if a sample contains 20% fibroblasts, 30% smooth muscle cells, and 50% endothelial cells, the column corresponding to that sample may contain entries as follows: [0.2 0.3 0.5] (where the column has been displayed as a row for convenience).

Let H be the matrix of gene expression profiles obtained from the samples of known composition. Each column in H corresponds to a sample. Each value in a column represents the expression level of a particular gene in the sample corresponding to that column. For example, if the expression levels of five genes are measured in three samples of known composition, then H will contain three columns, each containing five entries. The ith entry in the jth column represents the expression level of the ith gene in the sample corresponding to that column, i.e., the jth sample. Then, again assuming linearity:
P=HG⁻¹(Eq. 3)

Thus the matrix of pure cell type signatures, P, can be obtained from H and G, provided that G is invertible. If G is invertible, the solution for P can in general be found without requiring approximation. Note that when the composition of the samples can be selected, e.g., when the samples are prepared by mixing known proportions of pure cell populations, the entries in G are determined by the proportions selected. Thus G can be designed. Preferably G should be designed to have a small condition number, in order to obtain a stable solution to Eq. 3. According to certain embodiments of the invention the condition number is less than approximately 3. Preferably the condition number is less than approximately 2. More preferably the condition number is less than approximately 1.5. Yet more preferably the condition number is approximately 1.

It will be evident that the requirement that G is invertible means that the number of samples used to obtain G must equal the number of cell types that are present in the samples. In order to overcome this limitation, Eq. 3 can be modified so that G does not have to be invertible and can include the cell type composition of any number of known measured mixtures. In this case, H is multiplied by the pseudoinverse of G, and equation 3 will become:
P═HG^{T}(GG^{T})⁻¹ (Eq. 4)
where G^{T}(GG^{T})⁻¹ is the pseudoinverse of G and G^{T} is matrix G transposed. In this case G need not be a square matrix. In order for GG^{T} to be invertible, G should have maximal rank (which is the minimum of the number of columns and the number of rows of G). In this case this condition means that G should have rank equal to the number of different pure cell types (and also have that number of rows).

VII. Pure Cell Type Signature Databases

As described above, the invention provides a variety of ways to select a set of genes whose expression level defines a pure cell type signature for a cell type or cell state. According to certain embodiments of the invention information identifying the genes is stored in a database. The information may be stored in any suitable format sufficient to allow one of ordinary skill in the art to determine the identity of the genes. For example, the information may comprise accession numbers (e.g., GenBank accession numbers or accession numbers for any available gene database) and/or names of the genes or of expressed sequence tags (ESTs) derived from the genes.

Thus the invention provides a database stored on a computer-readable medium, wherein the database stores information for use in defining a pure cell type signature, the information comprising information identifying a set of genes whose expression level behaves in an approximately linear fashion across a plurality of mixed cell compositions in which cells of the first cell type are present at different percentages relative to other cell types present in the mixed cell compositions. According to certain embodiments of the invention the information comprises names and/or accession numbers of the genes and/or ESTs corresponding to the genes. According to certain embodiments of the invention the mixed cell compositions include at least one mixed cell composition in which more than 50% of the cells are cells of the first cell type and at least one mixed cell composition in which less than 50% of the cells are cells of the first type. According to certain embodiments of the invention the mixed cell compositions include at least one mixed cell composition that includes at least three different cell types.

The database may store information identifying genes for use in defining a plurality of pure cell type signatures. Each of the plurality of pure cell type signatures may correspond to a different cell type or cell state. The invention further provides a database such as those described above, further comprising expression levels for the set of genes, wherein the expression levels constitute a pure cell type signature for the first cell type. According to certain preferred embodiments of the invention the genes for use in defining a pure cell type signature exhibit consistent expression across a set of replicates.

The invention further provides a database stored on a computer-readable medium, wherein the database stores a pure cell type signature for a first cell type, the pure cell type signature comprising an expression level measured for each of a set of genes, wherein the genes are characterized in that their expression level behaves in an approximately linear fashion across a plurality of mixed cell compositions in which cells of the first cell type are present at different percentages relative to other cell types present in the mixed cell compositions. In addition to the expression levels themselves, the database typically includes information identifying the genes although this is not required. According to certain embodiments of the invention the mixed cell compositions include at least one mixed cell composition in which more than 50% of the cells are cells of the first cell type and at least one mixed cell composition in which less than 50% of the cells are cells of the first type. According to certain embodiments of the invention the mixed cell compositions include at least one mixed cell composition that includes at least three different cell types.

According to certain preferred embodiments of the invention the database stores a plurality of pure cell type signatures. Each of the plurality of pure cell type signatures may correspond to a different cell type or cell state. According to certain preferred embodiments of the invention the genes for use in defining a pure cell type signature exhibit consistent expression across a set of replicates.

The databases have a variety of uses. For example, once a set of genes suitable for use in defining a pure cell type signature has been identified, any individual who wishes to obtain a pure cell type signature under his or her own experimental conditions may make use of the information stored in the database that identifies genes suitable for defining a pure cell type signature. In addition, the database may be used to automatically select data for use in a pure cell type signature from any set of data that includes the expression levels of the genes identified in the database. Thus if microarray expression data for a particular cell type is available, the database facilitates automated extraction of expression levels for use in a pure cell type signature for that cell type. In general, the database of pure cell type signatures may be used to store and facilitate access to the pure cell type signature data used to practice the inventive methods of determining composition of a mixed cell population.

In particular, the invention provides a database stored on a computer-readable medium, wherein the database stores information identifying a set of genes for use in a pure cell type or cell state signature. In certain embodiments of the invention the genes comprise genes whose expression level behaves in an approximately linear fashion across a plurality of mixed cell compositions in which cells of the first cell type or cell state are present at different percentages relative to other cell types present in the mixed cell compositions. In certain embodiments of the invention the genes are characterized in that they exhibit consistent expression over a set of replicates. Any of the databases may further comprise expression levels for the set of genes, wherein the expression levels constitute pure cell type or state signatures.

VIII. Detection Methods and Technologies

Any of a variety of approaches may be used to obtain pure cell type specific signatures in accordance with the present invention. In general, gene expression can be measured at the RNA or protein level. When measuring gene expression at the RNA level, cDNA or oligonucleotide arrays, also known as microarrays, “GeneChips”, etc., provide a method of rapidly and efficiently measuring expression of a large number of genes.

cDNA microarrays consist of multiple (usually thousands) of different cDNAs spotted (usually using a robotic spotting device) onto known locations on a solid support, typically a rigid support such as a glass microscope slide. The cDNAs are typically obtained by PCR amplification of plasmid library inserts using primers complementary to the vector backbone portion of the plasmid or to the gene itself for genes where sequence is known. PCR products suitable for production of microarrays are typically between 0.5 and 2.5 kB in length. Full length cDNAs, expressed sequence tags (ESTs), or randomly chosen cDNAs from any library of interest can be chosen. ESTs are partially sequenced cDNAs as described, for example, in L. Hillier, et al., Generation and analysis of 280,000 human expressed sequence tags, Genome Research, 6, 807-828, 1996. Although some ESTs correspond to known genes, frequently very little or no information regarding any particular EST is available except for a small amount of 3′ and/or 5′ sequence and, possibly, the tissue of origin of the mRNA from which the EST was derived. As will be appreciated by one of ordinary skill in the art, in general the cDNAs contain sufficient sequence information to uniquely identify a gene within the human genome. Furthermore, in general the cDNAs are of sufficient length to hybridize, preferably specifically and yet more preferably uniquely, to cDNA obtained from mRNA derived from a single gene under the hybridization conditions of the experiment.

Oligonucleotide microarrays, in which oligonucleotides rather than cDNAs are employed to detect gene expression, represent an alternative to the use of cDNA microarrays (Lipshutz, R., et al., Nat Genet., 21(1 Suppl):20-4, 1999). In general, the experimental approach employed with an oligonucleotide microarray is similar to that used for cDNA microarrays. However, the shorter length of olignucleotides as compared with cDNAs means that care must be used to select oligonucleotides that hybridize specifically with transcripts whose level is to be measured. For purposes of description the invention will be described with reference to gene expression profiles obtained using cDNA microarrays rather than oligonucleotide microarrays, but it is to be understood that the latter could be used instead. Information regarding DNA microrarray technology and its applications may be found in Heller, M J, Annu Rev Biomed Eng., 4:129-53, 2002, and references cited therein. A variety of nucleic acid arrays have been developed and are known to those of skill in the art, including those described in: U.S. Pat. Nos. 5,242,974; 5,384,261; 5,405,783; 5,412,087; 5,424,186; 5,429,807; 5,436,327; 5,445,934; 5,472,672; 5,527,681; 5,529,756; 5,545,531; 5,554,501; 5,556,752; 5,561,071; 5,599,695; 5,624,711; 5,639,603; 5,658,734; WO 93/17126; WO 95/11995; WO 95/35505; EP 742 287; and EP 799 897.

In a typical microarray experiment, a microarray is hybridized with differentially labeled RNA or DNA populations derived from two different samples. Most commonly RNA (either total RNA or poly A⁺ RNA) is isolated from cells or tissues of interest and is reverse transcribed to yield cDNA. In general, one or more nucleotide residues is modified to include a label. In principle, the label may be directly or indirectly detectable. However, in many preferred embodiments, the label is a directly detectable label, by which is meant that it need not react with another chemical reagent or molecule in order to provide a detectable signal. One type of directly detectable label is an isotopic label, in which one or more of the nucleotides is labeled with a radioactive label, such as ³²S, ³²P, ³H, or the like. In yet other embodiments, light scattering particles may be employed as the label. Other sorts of labels that may be employed include various enzymatic labels, microparticles (e.g. quantum dots, nanocrystals, phosphors, etc.) See, e.g., Kricka L., Stains, labels and detection strategies for nucleic acids assays, Ann. Clin. Biochem., 39(2), pp. 114-129. According to certain embodiments of the invention a non-enzymatic method for RNA labeling is used, such as that described in Vineet, G., et al., Directly labeled mRNA produces highly precise and unbiased differential gene expression data, Nucleic Acids Research, 2003, Vol. 31, No. 4.

In many preferred embodiments, the directly detectable label is a fluorescent label. Fluorescent labels of interest (in various chemically conjugable forms) include: fluorescein, rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), the cyanine dyes, such as Cy3, Cy5, Alexa 542, Bodipy 630/650, fluorescent particles, fluorescent semiconductor nanocrystals, and the like. General discussion and comparison of various labeling methods employing fluorescent tags for use in cDNA and/or oligonucleotide microarray analysis (also applicable to other methods of analysis) is found in Richter, A., et al., Biotechniques, September;33(3):620-8, 630, 2002 and in Manduchi, E., et al., Physiol Genomics, September 3;10(3):169-79, 2002.

Labeling is frequently performed during reverse transcription by incorporating a labeled nucleotide in the reaction mixture. For example, the nucleotide may be conjugated with the fluorescent dyes Cy3 or Cy5. For example, Cy5-dUTP and Cy3-dUTP can be used. Alternately, an aminoallyl-labeled nucleotide such as aminoallyl-dUTP can be employed, and the aminoallyl group can be coupled with the label after reverse transcription. Other approaches include use of 3DNA structures (also known as dendrimers; available from Genisphere™) and hapten-antibody labeling.

In general, cDNA derived from one sample (representing, for example, a particular cell type, tissue type or growth condition) is labeled with one label (e.g., one fluor) while cDNA derived from a second sample (representing, for example, a different cell type, tissue type, or growth condition) is labeled with the second label (e.g., a second fluor). Similar amounts of labeled material from the two samples are cohybridized to the microarray. In the case of a microarray experiment in which the samples are labeled with Cy5(which fluoresces red) and Cy3(which fluoresces green), the primary data (obtained by scanning the microarray using a detector capable of quantitatively detecting fluorescence intensity) are ratios of fluorescence intensity (red/green, R/G). These ratios represent the relative concentrations of cDNA molecules that hybridized to the cDNAs represented on the microarray and thus reflect the relative expression levels of the mRNA corresponding to each cDNA/gene represented on the microarray. Although the description or microarrays presented herein refers primarily to methods involving two-color hybridizations, methods involving one-color or multi-color labeling may also be used. (See, e.g., U.S. Pat. No. 6,235,483).

The RNA may be amplified prior to or in conjunction with labeling. In general, any of a wide variety of amplification techniques known in the art can be used including, but not limited to, PCR, ligase chain reaction (LCR), rolling circle amplification, strand displacement amplification, etc. Certain of these methods may, optionally, be utilized for detection as well as amplification —for example by performing amplification directly on microarrays. See, e.g., Schweitzer, B. and Kingsmore, S., “Combining nucleic acid amplification and detection”, Curr Opin Biotechnol 2001 February;12(1):21-7, and references therein.

Preferably the amplification is linear, i.e., maintains the same relative proportions of different mRNA species as in the original sample. A variety of kits for performing linear amplification are commercially available, e.g., from Ambion (Austin, Tex.), Agilent and Arcturus (Mountain View, Calif.). Information regarding methods for performing linear amplification of RNA may be found in U.S. Pat. Nos. 5,514,545; 5,545,522; 5,716,785; 5,932,451; 6,132,997; and 6,235,483. See also US Patent Application Publication 20020110827, entitled “Quantitative mRNA Amplification”, filed December 21, to Hunter, et al. Amplification may be particularly advantageous when the sample contains only a small amount of RNA.

Each microarray experiment can provide tens of thousands of data points, each representing the relative expression of a particular gene in the two samples. Appropriate organization and analysis of the data is of great importance. Various computer programs that incorporate standard statistical tools have been developed to facilitate data analysis. One basis for organizing gene expression data is to group genes with similar expression patterns together into clusters. A method for performing hierarchical cluster analysis and display of data derived from microarray experiments is described in Eisen, M., Spellman, P., Brown, P., and Botstein, D., Cluster analysis and display of genome-wide expression patterns, Proc. Natl. Acad. Sci. USA, 95: 14863-14868, 1998. As described therein, clustering can be combined with a graphical representation of the primary data in which each data point is represented with a color that quantitatively and qualitatively represents that data point. By converting the data from a large table of numbers into a visual format, this process facilitates an intuitive analysis of the data. Additional information and details regarding the mathematical tools and/or the clustering approach itself may be found, for example, in Sokal, R. R. & Sneath, P. H. A. Principles of numerical taxonomy, xvi, 359, W. H. Freeman, San Francisco, 1963; Hartigan, J. A. Clustering algorithms, xiii, 351, Wiley, New York, 1975; Paull, K. D. et al. Display and analysis of patterns of differential activity of drugs against human tumor cell lines: development of mean graph and COMPARE algorithm. J Natl Cancer Inst 81, 1088-92,1989; Weinstein, J. N. et al. Neural computing in cancer drug development: predicting mechanism of action. Science 258, 447-51, 1992; van Osdol, W. W., Myers, T. G., Paull, K. D., Kohn, K. W. & Weinstein, J. N. Use of the Kohonen self-organizing map to study the mechanisms of action of chemotherapeutic agents. J Natl Cancer Inst 86, 1853-9, 1994; and Weinstein, J. N. et al. An information-intensive approach to the molecular pharmacology of cancer. Science, 275, 343-9, 1997. Additional approaches to processing, managing, and analyzing data obtained from microarray experiments are described in Pan, W., Bioinformatics, 18(4):546-54, 2002; Sherlock, G., Brief Bioinform, 2(4):350-62, 2001; Hess, K. R., Trends Biotechnol, 19(11):463-8, 2001. Such approaches may find use in conjunction with the present invention.

Further details of the experimental methods used in the present invention are found in the Examples. In particular, Example 1 describes the measurement of gene expression in pure cell populations using microarrays using a set of cDNA clones. It is noted that the validity of the approach described herein does not depend on the identity of the particular genes or clones whose expression is measured. The methods of the invention may be performed using any set of genes or clones, provided that the expression level of the genes or clones varies between the different cell types.

Additional information describing methods for fabricating and using microarrays is found in U.S. Pat. No. 5,807,522, which is herein incorporated by reference. Instructions for constructing microarray hardware (e.g., arrayers and scanners) using commercially available parts can be found at http://cmgm.stanford.edu/pbrown/ and in Cheung, V., Morley, M., Aguilar, F., Massimi, A., Kucherlapati, R., and Childs, G., Making and reading microarrays, Nature Genetics Supplement, 21:15-19, 1999, which are herein incorporated by reference. Additional discussions of microarray technology and protocols for preparing samples and performing microrarray experiments are found in, for example, DNA arrays for analysis of gene expression, Methods Enzymol, 303:179-205, 1999; Fluorescence-based expression monitoring using microarrays, Methods Enzymol, 306: 3-18, 1999; and M. Schena (ed.), DNA Microarrays: A Practical Approach, Oxford University Press, Oxford, UK, 1999. Descriptions of how to use an arrayer and the associated software are found at http://cmgm.stanford.edu/pbrown/mguide/arrayerHTML/ArrayerDocs.html, which is herein incorporated by reference.

Although microarrays represent a rapid and efficient means of measuring gene expression and obtaining expression profiles, in general, any measurement technique capable of determining RNA or protein presence or abundance may be used for these purposes. For RNA such techniques include, but are not limited to, Northern blots, RNAse protection assays, reverse transcription (RT)-PCR assays, real time RT-PCR (e.g., Taqman™ assay, Applied Biosystems), SAGE (Velculescu et al. Serial analysis of Gene Expression. Science, vol. 270, pp. 484487, October 1995), Invader® technology (Third Wave Technologies), etc. See, e.g., Eis, P. S. et al., Direct, sensitive quantitation of specific RNAs using an invasive cleavage assay. Nat. Biotechnol. 19:673(2001); Berggren, W. T. et al. Multiplexed gene expression analysis using the invader RNA assay with MALDI-TOF mass spectrometry detection. Anal. Chem. 74:1745(2002), etc. For proteins such techniques include, but are not limited to, immunoblots (Western blots), immunofluorescence, flow cytometry (e.g., using appropriate antibodies), mass spectrometry, protein microarrays (Elia, G., Trends Biotechnol 2002 December;20(12 Suppl):S19-22, and reference therein). As mentioned above, the invention encompasses the use of features such as RNA or protein modifications reflective of cell type or cell state. For example, the invention could make use of “protein modification state profiles” such as phosphorylation state profiles, etc. Appropriate detection methodologies for such states are known in the art. In addition, various array methodologies that differ from the microarrays described above may be used. For example, cDNAs can be arrayed on membranes or filters, which are then hybridized with probe and the signal quantified according to standard techniques.

IX. Implementation Systems and Methods

The present invention includes a computer system and software components for practicing the methods described above. The computer system can be a PC, workstation, etc., and is typically connected to one or more network lines or connections which can be part of an Ethernet link to other local computer systems, remote computer systems, or wide area communication networks, such as the Internet, etc.

A variety of software components will generally be loaded into memory during operation of the inventive system. These components function in concert to implement the methods described herein. The software components typically include an operating system and various languages and functions present on the system to enable execution of application programs that implement the inventive methods. Such components, include, for example, language-specific compilers, interpreters, and the like. Any of a wide variety of programming languages may be used to code the methods of the invention. Such languages include, but are not limited to, C, C++, JAVA™, etc. Typically the software components include a web browser.

In addition, the software components may include a mathematical/technical computing application program or package such as Matlab™ capable of performing matrix manipulations of the type described above in addition to a software application package representing the methods of the invention as embodied in a programming language of choice, which may be a special purpose language for use in conjunction with the application package. Typically the software components include a database program for storing and manipulating data, e.g., data from microarray experiments. The database may also store additional information such as pure cell type signatures for different cell types.

In an exemplary implementation, to practice the methods of the invention using such a computer system, a user provides data corresponding to a gene expression profile obtained from a mixed cell sample whose composition is to be determined to the computer, which data may then be loaded into memory. The data can be directly entered by the user or from other linked computer systems or on removable storage media, etc. For example, the computer system may be linked to an array scanner, and microarray data gathered by the scanner may be transferred directly to computer.

The software application package of the invention operates on the data to compute the cell type composition (vector q*) in the mixed cell sample. In accordance with the description above, in order to compute q*, the pure cell type signatures for the various cell types that may be present in the sample (i.e., the coefficients of P) must be available. The software components of the invention may include one or more lists of genes that may be used to define a pure cell type signature for each of a plurality of cell types. The user may then measure the expression levels for these genes using pure cell populations (or mixed populations of known composition), thereby determining the values for the pure cell type signatures. Alternately, or in addition, any of these software components may include values for the pure cell type signatures. The invention encompasses a process whereby pure cell type signatures may be developed for different tissues, different disease states, etc., and supplied to the user. The invention also encompasses a process whereby appropriate sets of genes for use in defining a pure cell type signature are developed over time and supplied to users who may then determine the values for the pure cell type signatures under their own laboratory conditions.

According to certain embodiments of the invention, the software components may request various items of information from the user and/or offer the user various options. For example, the user may be asked to enter information identifying the types of cells of interest. The user may be allowed to select to use one or more predetermined pure cell type signature(s) or to develop his/her own pure cell type signature(s). The user may make these selections using any of a number of methods, e.g., pull-down or pop-up menus, check boxes, radio buttons, fill in the blank, etc.

The description above has generally related to a system in which the user interacts directly with the computer that executes the application program encoding the methods of the invention. However, according to certain embodiments of the invention the system is implemented as a client/server system in which users enter information at a client computer, which information is then transmitted to a server computer that executes the application program. The client computer system can comprise any available computer but is typically a personal computer equipped with a processor, memory, display, keyboard, mouse, storage devices, appropriate interfaces for these components, and one or more network connections. According to these embodiments data (e.g., an expression profile obtained from a mixed cell sample) is entered at a client system and transmitted to a server system where the cell type composition of the sample is determined, and the resulting information is transmitted back to the client system. According to certain embodiments of the invention both the server and client computers are provided with software to support World Wide Web interactions.

Thus the invention provides a computer system for determining the cell type composition of a mixed cell population, wherein the mixed cell population contains cells of at least two cell types states, the computer system comprising: (a) memory means which stores a program comprising computer-executable process steps; and (b) a processor that executes the process steps so as (i) to receive data comprising a set of pure cell type or state signatures for cells in the mixed cell population; and (ii) to quantitatively determine the number, proportion, or relative number of cells of different cell types, cell states, or both, using the pure cell type or pure cell state signatures. According to certain embodiments of the invention the processor computes an approximate solution for one or more elements in a vector q, where q is a vector of quantities representing the number or proportion of cells of each cell type or cell state present in the mixed cell population, and wherein q satisfies the matrix equation Pq=m, where P is a matrix of pure cell type or pure cell state signatures. According to certain embodiments of the invention the processor computes a least squares solution for q. The memory may store a database of pure cell type or pure cell state signatures, such as those described above.

The invention further provides omputer-executable process steps stored on a computer-readable medium, the computer-executable process steps to quantitatively determine the number, proportion, or relative number of cells of different cell types, cell states, or both, in a mixed cell population, the computer-executable process steps comprising: (a) code to receive data comprising a set of pure cell type or pure cell state signatures for cells in the mixed cell population; and (b) code to quantitatively determine the number, proportion, or relative number of cells of different cell types, cell states, or both, in a mixed cell population using the expression profile. According to certain embodiments of the invention the code comprises code to compute an approximate solution for one or more elements in a vector q, where q is a vector of quantities representing the number or proportion of cells of each cell type or cell state present in the mixed cell population, and wherein q satisfies the matrix equation Pq=m, where P is a matrix of pure cell type or pure cell state signatures. In certain embodiments of the invention the code computes a least squares solution for q.

X. Applications

The methods and systems of the invention have a number of applications of which only a representative selection is presented here. In general, the methods of the invention are applicable for any of the myriad purposes for which gene expression of samples containing mixtures of cells is currently used or may be used in the future and expands the scope of applications for such technology by enhancing the specificity of the results. In particular, the ability to determine the cell type composition of mixed cell populations makes it possible to distinguish actual changes in gene expression of specific genes from differences in cellular composition, to determine the cellular composition of samples, and to detect the presence of specific cell types in samples.

A. Distinguishing True Changes in Gene Expression from Differences in Cellular Composition. The ability to determine cell type composition allows clinicians and researchers to distinguish differences in expression due to differences in the cellular content of samples versus true differences in gene expression levels in cells in the samples. The methods are particularly useful in contexts where differences in cellular composition can lead to “false positives”, i.e., an assessment that there has been an alteration in gene expression when in fact there has only been an alteration in cell composition or “false negatives”, i.e., a failure to detect an alteration in gene expression because of a compensating alteration in cell composition.

Differences in gene expression between normal and diseased tissue have been identified for many diseases. For example, differences in the gene expression profiles of normal and diseased blood vessels have been identified for numerous vascular diseases including atherosclerotic artery disease, peripheral artery disease, Takayusu's arteritis, giant cell arteritis, and systemic necrotizing vasculitis, etc. Differences in the gene expression profiles of normal cells and tumor cells of the same type have been identified for a large number of tumor types including breast cancer, lymphoma, leukemia, prostate cancer, colon cancer, melanoma, lung cancer, among others. In addition, differences between gene expression profiles of tumor cells in different subtypes of cancer have been identified, leading to the possibility of a molecular basis for cancer classification. See, e.g., Alizadeh A A, J Pathol, 195(1):41-52, 2002. Generally, establishing the existence of a difference in gene expression profile between normal and diseased tissues may involve analysis of numerous samples, careful examination of the samples (e.g., by a trained pathologist) to determine whether normal or diseased tissue is being analyzed, and possibly physical separation of normal and diseased tissue or of different cell types present within the sample prior to analysis. However, for purposes such as clinical diagnosis, available samples may be limited in size and will frequently include portions of both diseased and normal cells and/or mixtures of cell types. In general, it will be desirable to rapidly and reliably analyze the samples with a minimum of processing and minimal requirements for subjective interpretation.

Once the existence of a true difference in gene expression between normal and diseased cells is known, using this difference reliably for diagnosis is greatly facilitated by minimizing the effects of sample heterogeneity that can lead to false positives or negatives. In general, according to the methods herein a gene expression profile is obtained for a sample such as a biopsy specimen. The cell type composition of the sample is determined. If it is determined that the sample contains cells other than those whose gene expression pattern is altered in the disease state, an individual or a computer program interpreting the gene expression profile takes this information into account when interpreting the results. For example, the gene expression profile of the sample can be corrected, e.g., by subtracting the contribution of one or more cell types to the expression profile as described above. The corrected gene expression profile may then be meaningfully compared with known gene expression profiles for normal and/or diseased cells.

Differences in gene expression may be used not only for diagnosis or prognostication but also for monitoring response to treatment, monitoring exposure to toxic agents, radiation, pollutants, etc., as well as for basic research, e.g., biomedical research.

B. Identifying Cellular Composition. The ability to determine cell type composition is useful in a wide variety of areas. For example, expression profiling of samples from in vitro models of organ or tissue development can be used to detect the presence and relative ratios of specific cell types whose pure cell type signatures have been determined. This would allow monitoring of development of specific tissues in vitro or in vivo and would allow researchers and/or clinicians to assess the effects of specific treatments on these tissues. Once pure cell type signatures have been defined for normal cells and diseased cells, the methods described herein may be used to determine the proportion of normal cells versus diseased cells in tissue samples, which may be useful in assessing the severity of disease and/or response to therapy. The invention specifically contemplates use of the methods to determine the proportion of normal cells versus tumor cells in tumor tissue samples. For example, the proportion or number of endothelial cells in a tumor sample may be determined. Such a measurement allows the determination of the extent of vascularization or angiogenesis in a tumor based on the number, relative number, or proportion of endothelial cells. The effect of various treatments on tumor angiogenesis or vascularization may be ascertained by performing measurements at various time points following initiation of therapy.

C. Detection of Specific Cell Types. Establishment of pure cell type expression signatures and application of the methods described herein provides the ability to assay for the presence or absence of such cells in complex samples and to do so in a quantitative manner. For example, pure cell type expression signatures for vascular cells such as endothelial cells can be used to allow the detection of these cell types in, for example, tumor samples or tissue samples representing different stages of organ development. For tumor tissues this is particularly relevant for diagnostic, prognostic, therapeutic, and research purposes since aggressive tumor growth and metastases is dependent upon angiogenesis, i.e., the formation of new blood vessels in order to supply sufficient nutrients to the tumor cells and provide for gas exchange. Angiogenesis inhibitors are promising new agents for the treatment of cancer. The methods herein may be used to determine whether a particular tumor is a candidate for therapy using such agents and/or to monitor the efficacy of such treatment.

Other applications include the detection of vascular cells such as endothelial cells in diseases such as ischemic limb disease or angina, where therapeutic approaches (e.g. protein delivery, recombinant DNA) are attempting to induce angiogenesis in locations (e.g., limb and heart) where new vessel growth is required for normal tissue function. Yet another application is the detection of inflammatory monocyte/macrophage infiltration into tissue in autoimmune diseases and chronic inflammatory diseases including, but not limited to, systemic lupus erythematosus, Sjogren's syndrome, inflammatory bowel disease, rheumatoid arthritis, psoriasis, etc.

As another example, the methods may be used to determine whether a diagnostic sample is suitable for use in a diagnostic test. For instance, when attempting to diagnose lung infections, clinicians often attempt to obtain samples of sputum from the lungs. Patients are typically asked to expectorate, and sputum samples are cultured for the presence of bacteria. However, it is frequently the case that samples contain large proportions of material from the oral cavity, which makes them unsuitable for culture. Such contamination is detected by Gram staining and visually examining the specimen for the presence of epithelial cells. A large number of epithelial cells indicates that the specimen is not suitable for analysis. The methods of the present invention allows the quantitative detection of epithelial cells in such samples without the need for subjective interpretation. Similar approaches may be applied for other diagnostic tests. The ability to quantify sample composition will aid in the further standardization of diagnostic tests and improve their accuracy.

D. Determining Response to Treatment

A variety of treatments, including treatments for diseases, may result in an alteration in cell type or cell state. The invention is useful for detecting such alterations, and thereby assessing whether or not a cell population (or an individual from which a cell population has been obtained) has responded to a treatment and/or the extent of response. Thus the invention provides a method for determining whether cells of a given type or state in a cell population have responded to treatment comprising steps of: (a) quantitatively determining the number, relative number, or proportion of cells of different cell types or cell states using a first set of pure cell type or pure cell state signatures representing expression levels of genes whose expression does not change significantly under the treatment or stimulation, thereby obtaining the cell type or cell state composition of the sample; (b) calculating predicted expression levels using the cell type or cell state composition determined in step (a) and a second set of pure cell type or pure cell state signatures representing expression levels of genes whose expression does change significantly under treatment in cells of the given cell type or cell state; (c) measuring expression levels of the genes represented in the second pure cell type or state signature for cells of the given type in the cell population; (d) comparing the predicted expression levels and the measured expression levels; and (e) inferring that cells of the given cell type or cell state have responded to the treatment if the predicted and measured expression levels are sufficiently different. The treatment can be any kind of physical or chemical condition including, but not limited to, administration of pharmacologic agents such as drugs useful in treating disease. Thus the term “treatment” in the context of the foregoing method is not intended to limit the method.

The foregoing description is to be understood as being representative only and is not intended to be limiting. Alternative systems and methods for implementing the methods of the invention and also additional applications will be apparent to one of skill in the art, and are intended to be included within the accompanying claims. In particular, the accompanying claims are intended to include alternative program structures for implementing the methods of this invention that will be readily apparent to one of skill in the art.

### EXAMPLES

### Example 1

### Measuring Gene Expression in Pure Cell Populations Using Microarrays

Materials and Methods

Cells and Cell Culture. Human coronary artery endothelial cells (HCAEC, also referred to as EC), human coronary smooth muscle cells (HCASMC, also referred to as SMC), and human neonatal dermal fibroblast (FC) as well as cell-type defined culture medium were obtained from Clonefics, Inc. (San Diego, Calif.) at passage 3. Cells were cultured and maintained under standard conditions (37° C., 5% CO₂) in the appropriate cell-type defined medium with serum concentration as indicated by the manufacturer. Under these culture conditions, the cells were more than 99% pure. Purity was confirmed by Dil-Ac-LDL labeling of HCAEC as described in Netland, P. A., et al., In situ labeling of vascular endothelium with fluorescent acetylated low density lipoprotein, Histochemical Journal 17: 1309-1320, 1985. Cell type defined medium (Cambrex Corp., East Rutherford, N.J.) was as follows:

| Cells | Medium | Cat# |
|---|---|---|
| EC | EGM-2 MV Bulletkit System | CC-3202 |
| SMC | SmGM-2 Bulletkit System | CC-3182 |
| FC | FGM-2 Bulletkit System | CC-3132 |
| HeLa | DMEM with 10% bovine serum | |

To determine the response to stimulation, some of the cells were treated with 10 ng/ml of TNFα in the absence of serum for 24 hrs.

Cell Harvesting and RNA Isolation

Cells (EC, SMC, and FC) grown to passage 6 were harvested. Cells were harvested using Trypsin-Versene (EDTA) from Clonetics (Cat#: 17-161E). Total cell number of each cell type was counted by both Hemocytometer and Coulter Counter before extraction of RNA. RNA was extracted using a combination of Trizol (Life Technologies, Rockville, Md.) and RNAeasy column (Qiagen, Calif.) techniques according to the instructions of the manufacturer. Briefly, media was removed and two ml Trizol used per 3×10⁶ cells. Cells were sheared through a 21-gauge needle. The resulting solution was extracted with chloroform, and the supernatant mixed with 500 μl of 70% ethanol for every ml Trizol used initially. This mixture was then loaded and eluted from an RNAeasy column for further purification. RNA quality and concentration were evaluated by BioAnalyzer (Agilent Technologies, CA) and spectrophotometric analysis (OD260/280). RNA was prepared from HeLa cells in a similar manner.

cDNA Clone Selection and Microarray Construction

The cDNA microarrays were constructed from a total of 7476 DNA clones, which represented approximately 3900 different genes, including ESTs. 6528 clones were obtained from five vascular SMC libraries, and 288 clones from a TGF-β-treated endothelial cell library. All these libraries were cloned by suppression-subtraction hybridization. (Diatchenko, L. et al, Proc Natl Acad Sci USA 1996, 93: 6025-6030). The 5 SMC libraries were obtained from cells that had been stimulated with (i) TNF-α, (ii) TGF-β, (iii) PDGF-BB, (iv) stress; or (v) shear. 660 clones in the arrays were selected by performing virtual subtraction using expression data from public databases (the Unigene, the Serial Analysis of Gene Expression (SAGE) database at the NCBI (http://www.ncbi.nlm.nih.gov/SAGE/sagexpsetup.cgi), and BodyMap (http://bodymap.ims.u-tokvo.ac.jp/gene_ranking.php) (Hishiki, T., S. Kawamoto, S. Morishita, and K. Okubo. 2000. BodyMap: a human and mouse gene expression database. Nucleic Acids Res 28: 136-138.; Kawamoto, S., J. Yoshii, K. Mizuno, K. Ito, Y. Miyamoto, T. Ohnishi, R. Matoba, N. Hori, Y. Matsumoto, T. Okumura, Y. Nakao, H. Yoshii, J. Arimoto, H. Ohashi, H. Nakanishi, I. Ohno, J. Hashimoto, K. Shimizu, K. Maeda, H. Kuriyama, K. Nishida, A. Shimizu-Matsumoto, W. Adachi, R. Ito, S. Kawasaki, and K. S. Chae. 2000. BodyMap: a collection of 3′ ESTs for analysis of human gene expression information. Genome Res 10: 1817-1827) libraries) and were highly expressed in endothelial cells relative to other cell types. Briefly, the Library Differential Display feature of Unigene (http://www.ncbi.nlm.nih.gov/UniGene/ddd.cgi?ORG=Hs), the xProfiler tool of SAGE, and the Gene Ranking System of BodyMap were used to select genes that were differentially expressed in endothelial cell lines or endothelial tissue relative either to non-vascular cell lines, non-endothelial cell lines, or non-endothelial tissues. Various scoring metrics were employed to select those genes displaying the greatest differential expression, and genes having associated Unigene ID numbers were selected. Corresponding IMAGE clones were obtained from Research Genetics, Huntsville, Ala.

The clones were amplified by PCR employing flanking sequences of cloning vectors, according to standard methodology. Five μl of PCR reaction were visualized on 1% agarose gels for quality determination. PCR reactions were purified on a Qiagen BioRobot 3000. DNA microarrays were printed on glass slides employing Agilent's SurePrint ink-jet technology (Agilent Technologies, Inc., Palo Alto, Calif.). For a description of the performance features of Agilent's deposition cDNA microarrays with respect to uniformity, sensitivity, precision, and accuracy in gene expression profiling assays, see the Web site (having URL www.chem.agilent.com/scripts/LiteraturePDF.asp?iWHID=27667(visited Oct. 30, 2002 or www.chem.agilent.com/Scripts/PDS.asp?1Page=3449.

Sample Labeling, Microarray Hybridization, and Data Collection

In order to establish a mathematical model to allow the determination of the specific cell type composition of a sample containing a heterogeneous cellular population consisting of multiple cell types, sample RNAs from both pure cell type populations and mixed RNAs in different ratios from different cell types were labeled. At least two separate cultures of each cell type were employed for RNA preparation and hybridization. Total RNA from HeLa cells was used as a common reference for all the samples and labeled with Cy3-dye (green). Total RNAs from different cell samples were labeled with Cy5-dye (red channel). After labeling and before the Qiagen column purification of probes, Cy3- and Cy5-labeled products were mixed together.

The labeling and hybridization to the arrays was performed as follows. Briefly, ten μg of total RNA from cultured cells were reverse-transcribed in the presence of 400 units of Superscript II RNase H—Reverse Transcriptase (Invitrogen), 25 μM of dCTP and 100 μM each of dATP, dTTP and dGTP, 25 μM of Cy3- or Cy5-dCTP (NEN Life Science), 4 μM of 5′-T16N-3′ DNA primer and 27 units of RNase inhibitor (Amersham, N.J.). The labeling was carried out at 42° C. for 1 hour. After degradation of unlabeled RNA with RNase I, labeled cDNAs were purified with a Qiagen PCR cleanup kit according to the manufacturer's instructions. Microarray hybridization was performed at 65° C. overnight in a 25-μl of hybridization solution containing Agilent's deposition hybridization buffer, 5 units of PolydA₄₀₋₆₀, (Amersham, N.J.), 5 μg of yeast tRNA (Sigma, St. Louis, Mo.), 10 μg of human Cot 1 DNA (Invitrogen, Calif.) and Cy3- and Cy5-prelabeled HCV deposition control targets (Qiagen/Operon). At the end of hybridization, microarrays were first washed in 0.5×SSC/0.01% SDS for 5 min. at room temperature, and then washed in 0.06×SSC wash buffer for 10 min. Finally, microarrays were dried by centrifugation. The microarrays were scanned on Agilent's G2565AA Microarray Scanner System and the images were quantified using Agilent's G2567AA Feature Extraction Software Version A.5.1.1.

### Example 2

### Obtaining Pure Cell Type Signatures

Several different pure cell type signatures were developed for SMC, EC, and FC. Signature set 1(consisting of pure cell type signatures for SMC, EC, and FC) was generated by measuring the expression levels of all genes represented on the chip in pure cell populations of SMC, EC, and FC as described in Example 1. The expression levels were acquired by the scanner and imported into an Excel spreadsheet using Agilent Feature Extraction Software. The data were then converted to log ratios. The collection of expression levels for each cell type constituted the pure cell type signature for that cell type. The resulting spreadsheet was used as input to Matlab for computation of cell type composition of test samples containing different proportions of SMC, EC, and FC.

A second pure cell type signature set (signature set 2) that included genes whose expression was consistent among multiple replicates was developed as follows. Pure or mixed cell populations containing varying proportions of EC, SMC, or FC in ratios indicated in Table 1 were prepared by isolating RNA from different numbers (depending on the desired proportions) of cells from each pure cell populations and then mixing the RNA samples together. Four individual samples (replicates) corresponding to each of the ratios listed in Table 1 were prepared, resulting in a total of 40 samples. For each sample, the expression levels of all genes represented on the SMC chip were determined as described in Example 1. Genes with a variation of log ratio of <0.2 and a background-subtracted signal in the sample channel of more than 1000 but less than 20,000 among all 4 replicates for each of the ratios were considered to exhibit consistent expression and were selected for use in the pure cell type signatures for each cell type.

TABLE 1
Cell Proportion in Mixture

| EC | SMC | FC |
|---|---|---|
| 10 | 0 | 0 |
| 0 | 10 | 0 |
| 0 | 0 | 10 |
| 8 | 1 | 1 |
| 1 | 8 | 1 |
| 1 | 1 | 8 |
| 3 | 3 | 3 |
| 8 | 1 | 0 |
| 0 | 8 | 1 |
| 1 | 0 | 8 |

### Example 3

### Computing Cell Type Composition Using Pure Cell Type Signatures Consisting of 17 Genes Having Consistent Expression Across Replicates

This example describes the determination of the cell type composition of a sample using pure cell type signatures for EC, SCM, and FC in which the pure cell type signatures were based on 17 genes that exhibited consistent expression. Briefly, to obtain the pure cell type signatures, EC, SCM, and FC were cultured, harvested, and counted as described in Example 1. RNA was prepared and hybridized to a microarray and gene expression levels were measured as described in Example 1.

The pure cell type signatures represent expression levels of 17 genes represented on the microarray. The same methods are used for cell type signatures including larger numbers of genes. The 17 genes used in this example were selected because they were differentially expressed in all 3 cell types, i.e. any gene in this set has at least 0.25 difference in log ratio between any 2 cell types in pure cell samples. In addition, the expression of the genes was consistent across multiple replicates. Consistency of expression was determined as described below for the gene BG939384(caveolin 1, caveolae protein, 22 kD).

To demonstrate the ability of the inventive methods to determine the cell type composition of unknowns samples, test samples consisting of mixed cell populations containing known proportions of EC, SMC, and FC were prepared. Briefly, cells were cultured, harvested, and counted as described in Example 1. Cells were mixed in appropriate numbers to generate mixed cell compositions containing the various proportions of cells indicated in Table 2. For each composition, RNA was prepared and hybridized to a microarray, and gene expression levels were measured as described in Example 1.

Table 2 shows log ratio values measured for BG939384 for 7 different cell compositions, with 4 replicate experiments for each composition (i.e., the measurement was performed on 28 independently mixed samples). As is evident from Table 2, the log ratio of BG939384 for any given sample composition varied by less than 0.2 among all four replicates. Thus BG939384 exhibits consistent expression and is suitable for inclusion in a pure cell type specific signature in which genes having consistent expression are used.

TABLE 2

| Cell Proportion in Mixture | | | Log Ratios for Gene BG939384 | | | |
|---|---|---|---|---|---|---|
| Cell Type | | | Replicate | | | |
| EC | FC | SMC | 1 | 2 | 3 | 4 |
| 10 | 0 | 0 | 1.33 | 1.27 | 1.36 | 1.35 |
| 0 | 0 | 10 | 0.26 | 0.35 | 0.30 | 0.24 |
| 0 | 10 | 0 | 1.07 | 1.10 | 1.04 | 1.03 |
| 8 | 1 | 1 | 1.25 | 1.27 | 1.16 | 1.24 |
| 1 | 1 | 8 | 0.70 | 0.68 | 0.69 | 0.67 |
| 1 | 8 | 1 | 1.04 | 1.04 | 0.99 | 1.02 |
| 3 | 3 | 3 | 1.03 | 1.01 | 1.08 | 1.04 |

The log ratios were averaged across all replicates for each cell type composition for each gene. Table 3 shows average log ratio data for 17 selected genes and 7 different experiments (3 pure cell samples, 4 mixtures with different proportions of cells). In the top row of the table, the headings EC, SMC, and FC indicate pure cell populations and the headings that list proportions represent mixtures of EC:SMC:FC. The accession numbers represent hits that were found when sequences from the clones were used to search GenBank.

TABLE 3

| Accession | | | | | | | | |
|---|---|---|---|---|---|---|---|---|
| number | Lab ID | EC | SMC | FC | [8:1:1] | [1:8:1] | [1:1:8] | [3:3:3] |
| No hits found | 9F.6.G4 | 1.38 | 0.29 | 1.11 | 1.27 | 0.70 | 1.06 | 1.07 |
| No hits found | 9R.5.G4 | −0.13 | 0.55 | 0.84 | 0.22 | 0.59 | 0.82 | 0.61 |
| BG150376 | 9R.6.C1 | −0.11 | 0.23 | −0.36 | −0.09 | 0.14 | −0.25 | −0.03 |
| BG819442 | 11R.1.F2 | −0.25 | 0.26 | 0.00 | −0.19 | 0.17 | −0.06 | −0.01 |
| BG715344 | 9R.6.D5 | 1.17 | 1.42 | 0.61 | 1.13 | 1.29 | 0.86 | 1.13 |
| BG771368 | 11R.1.D3 | 0.63 | 0.88 | 0.25 | 0.59 | 0.79 | 0.41 | 0.64 |
| AF186409 | 1R.1.D6 | 0.87 | 0.58 | −0.03 | 0.80 | 0.57 | 0.29 | 0.59 |
| BC012527 | 12R.1.G7 | 0.05 | 0.33 | −0.21 | 0.02 | 0.24 | −0.18 | 0.05 |
| No hits found | 9R.5.A7 | −0.13 | 0.54 | 0.84 | 0.21 | 0.57 | 0.80 | 0.61 |
| AI472137 | 9F.4.D10 | 1.11 | 0.48 | −0.27 | 1.00 | 0.51 | 0.27 | 0.70 |
| AF132203 | 8F.7.A12 | −0.45 | 0.01 | 0.48 | −0.18 | 0.09 | 0.34 | 0.14 |
| AI718771 | 7R.4.B12 | −0.12 | 0.53 | 0.81 | 0.19 | 0.57 | 0.78 | 0.57 |
| BG542672 | 7F.3.C9 | 1.33 | 0.66 | −0.48 | 1.24 | 0.76 | 0.46 | 0.94 |
| AU138027 | 7F.6.H12 | 1.08 | 0.50 | −0.22 | 1.02 | 0.58 | 0.34 | 0.74 |
| BG533142 | 7R.8.H12 | 0.93 | 0.64 | 0.03 | 0.82 | 0.63 | 0.28 | 0.62 |
| BG939384 | 9R.2.B9 | 1.33 | 0.29 | 1.06 | 1.23 | 0.69 | 1.02 | 1.04 |
| No hits found | 8R.10.H12 | 1.23 | 0.59 | −0.45 | 1.17 | 0.68 | 0.40 | 0.87 |

For these genes the matrix P of pure cell type signatures consists of the actual ratios corresponding to the third, fourth, and fifth columns from the table above (i.e. 10 to the power of the corresponding entry). These ratios are shown in Table 4A.

TABLE 4A

| Accession number | Lab ID | EC | SMC | FC | [8:1:1] | [1:8:1] | [1:1:8] | [3:3:3] |
|---|---|---|---|---|---|---|---|---|
| No hits found | 9F.6.G4 | 23.99 | 1.95 | 12.88 | 18.62 | 5.01 | 11.48 | 11.75 |
| No hits found | 9R.5.G4 | 0.74 | 3.55 | 6.92 | 1.66 | 3.89 | 6.61 | 4.07 |
| BG150376 | 9R.6.C1 | 0.78 | 1.70 | 0.44 | 0.81 | 1.38 | 0.56 | 0.93 |
| BG819442 | 11R.1.F2 | 0.56 | 1.82 | 1.00 | 0.65 | 1.48 | 0.87 | 0.98 |
| BG715344 | 9R.6.D5 | 14.79 | 26.30 | 4.07 | 13.49 | 19.50 | 7.24 | 13.49 |
| BG771368 | 11R.1.D3 | 4.27 | 7.59 | 1.78 | 3.89 | 6.17 | 2.57 | 4.37 |
| AF186409 | 1R.1.D6 | 7.41 | 3.80 | 0.93 | 6.31 | 3.72 | 1.95 | 3.89 |
| BC012527 | 12R.1.G7 | 1.12 | 2.14 | 0.62 | 1.05 | 1.74 | 0.66 | 1.12 |
| No hits found | 9R.5.A7 | 0.74 | 3.47 | 6.92 | 1.62 | 3.72 | 6.31 | 4.07 |
| AI472137 | 9F.4.D10 | 12.88 | 3.02 | 0.54 | 10.00 | 3.24 | 1.86 | 5.01 |
| AF132203 | 8F.7.A12 | 0.35 | 1.02 | 3.02 | 0.66 | 1.23 | 2.19 | 1.38 |
| AI718771 | 7R.4.B12 | 0.76 | 3.39 | 6.46 | 1.55 | 3.72 | 6.03 | 3.72 |
| BG542672 | 7F.3.C9 | 21.38 | 4.57 | 0.33 | 17.38 | 5.75 | 2.88 | 8.71 |
| AU138027 | 7F.6.H12 | 12.02 | 3.16 | 0.60 | 10.47 | 3.80 | 2.19 | 5.50 |
| BG533142 | 7R.8.H12 | 8.51 | 4.37 | 1.07 | 6.61 | 4.27 | 1.91 | 4.17 |
| BG939384 | 9R.2.B9 | 21.38 | 1.95 | 11.48 | 16.98 | 4.90 | 10.47 | 10.96 |
| No hits found | 8R.10.H12 | 16.98 | 3.89 | 0.35 | 14.79 | 4.79 | 2.51 | 7.41 |

In order to account for the fact that 10 μg of total RNA was used in each pure cell reaction, we multiply P by the inverse to the matrix K, where:

$$K = \begin{matrix}10 & 0 & 0 \\ 0 & 10 & 0 \\ 0 & 0 & 10\end{matrix}$$

$$\text{which is:}$$

$${K}^{- 1} = \begin{matrix}0.1 & 0 & 0 \\ 0 & 0.1 & 0 \\ 0 & 0 & 0.1\end{matrix}$$

The multiplication is performed in order to convert the numbers in P into expression signatures of unit quantities of cells (i.e., the unit quantity is 1 ug rather than 10 ug. The result is shown in Table 4B, in which the second, third, and fourth columns are the standardized matrix of cell type signatures of pure cells, Pₛ=(P)(K⁻¹).

TABLE 4B

| Accession | | | | |
|---|---|---|---|---|
| number | Lab ID | EC | SMC | FC |
| No hits found | 9F.6.G4 | 2.40 | 0.19 | 1.29 |
| No hits found | 9R.5.G4 | 0.07 | 0.35 | 0.69 |
| BG150376 | 9R.6.C1 | 0.08 | 0.17 | 0.04 |
| BG819442 | 11R.1.F2 | 0.06 | 0.18 | 0.10 |
| BG715344 | 9R.6.D5 | 1.48 | 2.63 | 0.41 |
| BG771368 | 11R.1.D3 | 0.43 | 0.76 | 0.18 |
| AF186409 | 1R.1.D6 | 0.74 | 0.38 | 0.09 |
| BC012527 | 12R.1.G7 | 0.11 | 0.21 | 0.06 |
| No hits found | 9R.5.A7 | 0.07 | 0.35 | 0.69 |
| AI472137 | 9F.4.D10 | 1.29 | 0.30 | 0.05 |
| AF132203 | 8F.7.A12 | 0.04 | 0.10 | 0.30 |
| AI718771 | 7R.4.B12 | 0.08 | 0.34 | 0.65 |
| BG542672 | 7F.3.C9 | 2.14 | 0.46 | 0.03 |
| AU138027 | 7F.6.H12 | 1.20 | 0.32 | 0.06 |
| BG533142 | 7R.8.H12 | 0.85 | 0.44 | 0.11 |
| BG939384 | 9R.2.B9 | 2.14 | 0.19 | 1.15 |
| No hits found | 8R.10.H12 | 1.70 | 0.39 | 0.04 |

Consider a vector that corresponds to the results of the measurements for one of the mixtures, e.g. for the measured expression of endothelial cells, smooth muscle cells and fibroblast cells in proportions 8:1:1. This vector will be referred to as m, and is given by the third column in Table 5, which is identical to the sixth column in Table 4A:

TABLE 5

| Accession | | |
|---|---|---|
| number | Lab ID | [8:1:1] |
| No hits found | 9F.6.G4 | 18.62 |
| No hits found | 9R.5.G4 | 1.66 |
| BG150376 | 9R.6.C1 | 0.81 |
| BG819442 | 11R.1.F2 | 0.65 |
| BG715344 | 9R.6.D5 | 13.49 |
| BG771368 | 11R.1.D3 | 3.89 |
| AF186409 | 1R.1.D6 | 6.31 |
| BC012527 | 12R.1.G7 | 1.05 |
| No hits found | 9R.5.A7 | 1.62 |
| AI472137 | 9F.4.D10 | 10.00 |
| AF132203 | 8F.7.A12 | 0.66 |
| AI718771 | 7R.4.B12 | 1.55 |
| BG542672 | 7F.3.C9 | 17.38 |
| AU138027 | 7F.6.H12 | 10.47 |
| BG533142 | 7R.8.H12 | 6.61 |
| BG939384 | 9R.2.B9 | 16.98 |
| No hits found | 8R.10.H12 | 14.79 |

Now we will solve the equation Pₛq=m, where q is the unknown vector of mixtures, using the least squares algorithm, which minimizes norm(m−Pₛq) as described above (see Golub, referenced above). The Matlab software package has standard functions lsqr( ) and lsqnormeg( ) that implement a least squares algorithm for solving this type of equation. The latter function finds a solution q* with nonnegative coefficients, which is appropriate and was used in this case. Applying the lsqnormeg( ) function with parameters Pₛ and m yields q*=[8.75 0.81 0.23]. Thus we predict that the sample obtained from the mixed cell population contains 8.75 ug EC RNA, 0.81 ug SMC RNA, and 0.23 ug FC RNA.

Applying the same procedure described above to test three additional samples of known compositions 1:8:1, 1:1:8, and 3:3:3 produces the results in Table 6, in which the “Known” columns represent the known cell type composition of the test samples, and the “Found” columns represent the predicted values derived by applying the methods of the invention to expression data obtained from test samples of known cell type composition. Results in the “Found” columns of Table 6 are normalized to 10, in order to account for the fact that 10 ug of total RNA was used in each reaction with known composition.

TABLE 6

| Known | | | Found | | |
|---|---|---|---|---|---|
| EC | SMC | FC | EC | SMC | FC |
| 8 | 1 | 1 | 8.83 | 0.91 | 0.26 |
| 1 | 8 | 1 | 1.12 | 7.41 | 1.47 |
| 1 | 1 | 8 | 0.79 | 1.28 | 7.93 |
| 3 | 3 | 3 | 3.43 | 3.17 | 3.40 |

These results demonstrate that the methods of the invention may be used to accurately determine the cell type composition of mixed cell samples of unknown cell type composition.

### Example 4

### Computing Cell Type Composition Using Pure Cell Type Signatures Consisting of Genes Having Consistent Expression Across Replicates

This example describes the determination of the cell type composition of a sample using pure cell type signatures for EC, SCM, and FC in which the pure cell type signatures were based on a larger set of genes that exhibited consistent expression, i.e., all genes represented on the microarray that exhibited consistent expression. Briefly, to obtain the pure cell type signatures, EC, SCM, and FC were cultured, harvested, and counted as described in Example 1. RNA was prepared and hybridized to a microarray and gene expression levels were measured as described in Example 1.

To demonstrate the ability of the inventive methods to determine the cell type composition of unknowns samples, test samples consisting of mixed cell populations containing known proportions of EC, SMC, and FC were prepared. Briefly, cells were cultured, harvested, and counted as described in Example 1. Cells were mixed in appropriate numbers to generate mixed cell compositions containing the various proportions of cells indicated in Table 7. For each composition, RNA was prepared and hybridized to a microarray and gene expression levels were measured as described in Example 1. The expression levels for each sample constituted the values for the vector m for that sample and were used as input to the computer program described above (a Matlab routine) that computed the least squares solution q* for the equation Pq=m using a matrix P of pure cell signatures based on genes that exhibited consistent expression, where consistent genes were genes whose log ratio varied by less than 0.2 among four replicates where the background-subtracted signal in the sample channel was more than 1000 but less than 20,000. Thus q* contained an entry corresponding to each cell type, which represented the proportion of cells of that type in the sample.

Table 7 presents the known proportions of the samples (Known) and solutions for their composition as determined by solving for q (Found). As is evident from Table 7, the solutions closely matched the known composition of the sample.

TABLE 7

| Known | | | Found | | | |
|---|---|---|---|---|---|---|
| EC | SMC | FC | EC | SMC | FC | Error |
| 8 | 1 | 1 | 7.89 | 0.96 | 0.96 | 0.13 |
| 1 | 8 | 1 | 0.96 | 7.69 | 0.97 | 0.32 |
| 1 | 1 | 8 | 0.92 | 0.97 | 7.71 | 0.32 |
| 3 | 3 | 3 | 3.16 | 3.16 | 3.18 | 0.29 |
| 8 | 1 | 0 | 9.09 | 0.73 | 0.17 | 1.14 |
| 0 | 8 | 1 | 0.00 | 9.73 | 0.70 | 1.76 |
| 1 | 0 | 8 | 1.27 | 0.00 | 8.50 | 0.57 |

### Example 5

### Computing Cell Type Composition Using Pure Cell Type Signatures Consisting of an Unbiased Set of Genes

This example describes the determination of the cell type composition of a sample using pure cell type signatures for EC, SCM, and FC in which pure cell type signatures were based on all genes represented on the microarray rather than only a subset that exhibited consistent expression. Briefly, to obtain the pure cell type signatures, EC, SCM, and FC were cultured, harvested, and counted as described in Example 1. RNA was prepared and hybridized to a microarray and gene expression levels were measured as described in Example 1.

To demonstrate the ability of the inventive methods to determine the cell type composition of unknowns samples, test samples consisting of mixed cell populations containing known proportions of EC, SMC, and FC were prepared. Briefly, cells were cultured, harvested, and counted as described in Example 1. Cells were mixed in appropriate numbers to generate mixed cell compositions containing the various proportions of cells indicated in Table 8. For each composition, RNA was prepared and hybridized to a microarray and gene expression levels were measured as described in Example 1. The expression levels for each sample constituted the values for the vector m for that sample and were used as input to the computer program described above (a Matlab routine) that computed the least squares solution q* for the equation Pq=m using the matrix P of pure cell type signatures based on all genes. Thus q* contained an entry corresponding to each cell type, which represented the proportion of cells of that type in the sample.

Table 8 presents the known proportions of the samples (Known) and solutions for their composition as determined by solving for q (Found). As is evident from Table 8, the solutions approximated the known composition of the sample. However, it is noted that the results in this case were inferior to experiments in which genes were preselected (e.g., for consistency).

TABLE 8

| Known | | | Found | | | |
|---|---|---|---|---|---|---|
| EC | SMC | FC | EC | SMC | FC | Error |
| 8 | 1 | 1 | 5.87 | 3.39 | 1.20 | 3.21 |
| 1 | 8 | 1 | 1.18 | 8.29 | 0.91 | 0.35 |
| 1 | 1 | 8 | 1.20 | 3.62 | 5.73 | 3.47 |
| 3 | 3 | 3 | 2.51 | 5.67 | 2.42 | 2.77 |
| 8 | 1 | 0 | 6.55 | 3.41 | 0.00 | 2.81 |
| 0 | 8 | 1 | 0.09 | 8.82 | 1.33 | 0.89 |
| 1 | 0 | 8 | 1.60 | 0.79 | 7.56 | 1.09 |

### Example 6

### Computing Cell Type Composition in an Arterial Wall Biopsy

Atherosclerosis, a process involving lipid deposition and smooth muscle cell (SMC) proliferation in the vascular wall, can affect various organs and regions depending on the affected vascular bed. Atherosclerotic coronary artery disease, i.e., the focal narrowing of larger and medium sized coronary arteries characterized by proliferation of SMCs and the deposition of lipids, is now the leading cause of death in the developed world. The molecular mechanisms underlying atherosclerosis are not fully understood.

The normal vascular wall of arteries and veins consists of three layers. The intima, lined by a monolayer of endothelial cells (EC) in contact with blood, contains resident SMC embedded in extracellular matrix. The internal elastic lamina forms the border of the intima with the underlying tunica media, which contains layers of SMC. The SMC, EC and FC are the major cell types in the vascular wall. The proportion of cell types varies widely in different regions of arteries and may also vary among different arteries. In general, the SMC is the most abundant cell type in the arterial wall. EC play a very important role in vascular physiology despite the fact that their relative numbers are relatively small. ECs form a monolayer along the interior of the vessel wall, so that in general their numbers are roughly constant when measured per surface area of vessel in normal samples and samples from both diseased vessels.

The development of atherosclerosis may involve lipoprotein deposition and leukocyte recruitment in the arterial wall. The initiation of atherosclerosis may begin with accumulation and modification of lipoprotein in the intima of the arterial wall, increased permeability (leakiness) of the endothelium, and an increased collection of intima involving changes in the extracellular matrix, eventually leading to atheroma (plaque) formation. Atheroma evolution involves SMC. During atherogenesis, the arterial wall undergoes dramatic remodeling. Cytokines and growth factors such as PDGF and TBFβ, etc., released by vascular cells and infiltrating leukocytes are believed to stimulate SMC proliferation, and focal vascular wall inflammation leads to luminal narrowing and occlusive thrombus formation. SMC numbers may vary along the length of a vessel, which may contribute to focal differences.

Vascular cells and activated macrophages in the lesion may modulate inhibition of atheroma through various molecular signaling mechanisms. In order to study these cellular interactions and to determine the effects of various treatments on the processes involved in atherogenesis, a culture system is established in which EC, SMC, and FC are cultured together in vitro. The culture is exposed to various treatments (e.g., cytokines and growth factors) and gene expression profiles are obtained using microarray analysis as described in Example 1. In addition, samples are obtained from arterial walls in which atheroma is present.

In order to determine whether the treatments mimic the process of atherogenesis that occurs in vivo, gene expression profiles obtained from the arterial wall samples are compared with gene expression profiles obtained from cells in the culture system. To determine whether the treatments result in true changes in gene expression (e.g., shifting the gene expression profile of the cell in culture so that it more closely resembles the gene expression profile found in diseased arterial wall), or whether they are due to alterations in cell type composition, it is necessary to determine the relative contributions of cells of each type. Therefore, the cell type composition of the arterial wall samples and the cell type composition of mixed cell populations grown in tissue culture are determined using pure cell type expression signatures as described in Examples 3, 4, 5, and 6. The gene expression profiles obtained from the cultures are normalized so that the expression levels of specific genes in the arterial wall samples may be compared with the expression level in the samples obtained from tissue culture. Such comparisons may be performed for each cell type.

This process allows the refinement of the in vitro culture system to more closely replicate the in vivo situation, resulting in an in vitro model that can be used for a variety of purposes. For example, the system may be used to determine which cytokines and growth factors are likely to play a role in atherogenesis, to identify genes whose expression is affected by such agents, and also to determine which cells alter their gene expression profiles in response to such agents. In contrast to systems in which each cell type is cultured individually, the system described herein allows the effects of cell-cell interactions to be to determined. For example, if an agent stimulates EC to release factors that alter gene expression in SMC, such an effect can be detected using a mixed cell culture system whereas it would not be possible to detect such an effect using single cell type culture systems. Determining the cell type composition of the tissue culture samples allows the identification of agents (e.g., cytokines and/or growth factors) that selectively stimulate SMC proliferation, which is an important contributor to atherogenesis, as opposed to agents that stimulate cell proliferation in general. Inhibition of these agents may be an appropriate therapeutic or preventive strategy for atherosclerosis.

Determination of cell type composition can also be used to more accurately assess the effects of various potential therapies on the process of atherogenesis using an animal model. The inbred transgenic atherosclerosis-polygenic hypertension Dahl salt-sensitive (S) rat model (Tg53) over-expresses human cholesteryl ester transfer protein (hCETP) in the liver and exhibits coronary artery disease and decreased survival compared with control non-transgenic Dahl S rats (Herrera, V M, Mol. Med., 7(12):831-44, 2001). Tg53 and nontransgenic counterparts rats are maintained under standard laboratory conditions and fed a standard diet. Thirty adult TG53 rats and thirty nontransgenic animals are divided into 6 groups consisting of 10 animals each (5 Tg53 and 5 nontransgenic). A different candidate therapeutic agent is administered to each of 5 groups with the 6^{th} group serving as a control (no agent administered).

Arterial biopsies are obtained after a treatment period of appropriate length (e.g., 6 weeks), and gene expression profiles are determined using microarray analysis. The percentages of SMC, EC, and FC in each sample are determined using pure cell type expression signatures as described in Example 3. Using the cell type compositions, the contribution of each cell type to the expression level of each gene is determined, and expression profiles are normalized so that alterations in actual gene expression in any of the cell types are detected. The effects of the different treatments on both cell type composition and gene expression levels in each cell type are compared. Treatments that result in either a cell type composition that more closely resembles normal cell type composition and/or a gene expression profile that more closely resembles that observed in the samples from normal rats are identified as potential therapeutic or preventive agents for atherosclerosis.

In addition to assessing the effect of the various treatments on the relative numbers of SMC, EC, and FC and on gene expression levels, the presence, relative number, and activation state of macrophages in the arterial biopsies is determined by including pure cell type signatures for unactivated and activated macrophages in the matrix P of pure cell type signatures.

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

1. A method of analyzing a mixed cell population comprising the steps of:
providing or determining a pure cell type or pure cell state signature for cells of different cell types or states in the mixed cell population; and
quantitatively determining the number, proportion, or relative number of cells of different cell types or cell states in the mixed cell population using the pure cell type signatures or pure cell state signatures for the cell types or cell states.

2. The method of claim 1, wherein the step of quantitatively determining comprises solving a matrix equation that relates the pure cell type or pure cell state signatures to gene expression levels measured in the mixed cell population.

3. The method of claim 1, wherein the mixed cell population comprises cells of at least three cell types or cell states.

4. The method of claim 1, wherein the pure cell type or pure cell state signature of at least one cell type or cell state comprises at least 10 genes.

5. The method of claim 1, wherein the mixed cell population comprises cells that are or were infected by an infectious agent.

6. The method of claim 4, wherein at least one pure cell type or pure cell state signature includes one or more genes that occur in the genome of the infectious agent.

7. The method of claim 1, wherein the mixed cell population comprises exposed and unexposed cells.

8. The method of claim 1, wherein the mixed cell population comprises normal cells and cells in a diseased state.

9. The method of claim 1, wherein the cells in the mixed cell population comprise tumor cells or endothelial cells.

10. The method of claim 1, wherein the cells in the mixed cell population comprise endothelial cells and tumor cells from a tumor, further comprising the step of:
determining the extent of vascularization or angiogenesis in the tumor based on the number, relative number, or proportion of endothelial cells.

11. The method of claim 1, wherein the cells in the mixed cell population comprise one or more cell types selected from the group consisting of: fibroblasts, endothelial cells and smooth muscle cells, or wherein the mixed cell population comprises cells from a sample obtained from a blood vessel wall.

12. The method of claim 1, wherein the step of quantitatively determining the number, proportion, or relative number of cells of different cell types or cell states in the mixed cell population comprises:
computing an approximate solution for one or more elements in a vector q, where q is a vector of quantities representing the number, relative number, or proportion of cells of each cell type or cell state present in the mixed cell population, and wherein q satisfies the matrix equation Pq=m, where P is a matrix of pure cell type or pure cell state signatures and m is a vector of quantities representing expression levels of genes included in the pure cell type or cell state signatures in the mixed cell population.

13. The method of claim 12, wherein the signature elements of the pure cell type or cell state signatures are mRNA expression levels of a set of genes, which are optionally measured using a microarray, or wherein the signature elements of the pure cell type or cell state signatures are protein expression levels of a set of genes.

14. The method of claim 12, wherein q represents the number of cells of each cell type or cell state present in the mixed cell population.

15. The method of claim 12, wherein q is expressed in terms of unit quantities of cells.

16. The method of claim 12, wherein:
the computing step comprises computing a least squares solution.

17. The method of claim 12, wherein the signature elements of the pure cell type or pure cell state signatures are expression levels of genes, and wherein the vector m represents expression levels of all genes included in the pure cell type or pure cell state signatures.

18. The method of claim 12, wherein the signature elements of the pure cell type or pure cell state signatures are expression levels of genes, and wherein the vector m represents expression levels of some of the genes included in the pure cell type or pure cell state signatures.

19. The method of claim 12, wherein:
the columns of the matrix of pure cell type or pure cell state signatures comprise expression levels of a set of genes in a pure cell population.

20. The method of claim 12 wherein:
the matrix of pure cell type or pure cell state signatures is obtained by measuring expression levels of a set of genes in at least one mixed cell population in which the number, relative number, or proportion of cells of different cell types or cell states is known and inferring expression levels of the set of genes in pure cell populations from the expression levels of the genes in the at least one mixed cell population.

21. The method of claim 12, wherein:
the set of genes consists entirely or substantially of genes that are differentially expressed between the cell types or cell states.

22. A method of defining a pure cell type or pure cell state signature comprising steps of:
providing a population of cells;
obtaining an expression profile for the population of cells across a set of genes, the set comprising at least 10 genes;
repeating the providing and obtaining steps at least once using different populations of cells, thereby generating results for at least two replicates; and
selecting genes whose expression is consistent among the replicates for use in the pure cell type signature.

23. The method of claim 22, wherein the set of genes comprises at least 100 genes, at least 1000 genes, or at least 5000 genes.

24. The method of claim 22, wherein the populations of cells include at least one pure cell population.

25. The method of claim 22, wherein the populations of cells include at least one mixed cell population of known composition.

26. A method of defining a pure cell type or pure cell state signature comprising steps of:
performing the method of claim 22 a plurality of times, wherein each initial providing step comprises providing either a pure cell population or a mixed cell population of known composition; and
selecting genes whose expression is consistent among all the replicates provided in each repetition of the method of claim 22 for use in the pure cell type or pure cell state signature.

27. A method of defining a pure cell type or pure cell state signature comprising steps of:
providing a population of cells;
obtaining an expression profile for the population of cells across a set of genes, the set comprising at least 10 genes;
repeating the providing and obtaining steps at least once using different populations of cells having a range of known cell type or cell state compositions; and
selecting genes whose expression level behaves in a linear fashion across the range of cell type or cell state compositions for use in the pure cell type or pure cell state signature.

28. The method of claim 27, wherein the set of genes comprises at least 100 genes, at least 1000 genes, or at least 5000 genes.

29. A method of determining a response to treatment or stimulation comprising steps of:
providing a sample comprising a population of cells, wherein some or all of the cells have responded to a treatment or stimulation and wherein response to a treatment or stimulation leads to a change in expression of at least one gene;
providing or determining a pure cell type or pure cell state signature for cells of at least two different cell types or cell states in the mixed cell population, wherein the pure cell type or pure cell state signatures include a cell type or cell state signature for a cell that has responded to the treatment or stimulation and a pure cell type or pure cell state signature for a cell that has not responded to the treatment or stimulation; and
quantitatively determining the number, proportion, or relative number of cells in the mixed cell population that have responded to the treatment or stimulation using the pure cell type or pure cell state signatures.

30. The method of claim 29, wherein the sample comprises a cell or tissue sample obtained from a subject who has been exposed to the treatment or stimulation.

31. The method of claim 29, wherein the cells are obtained from a subject, further comprising the step of:
determining that the subject has responded to the treatment or stimulation if the number, relative number, or proportion of cells that have responded to the treatment or stimulation exceeds a predetermined value.

32. The method of claim 29, wherein the quantitatively determining step comprises:
computing an approximate solution for one or more elements in a vector q, where q is a vector of quantities representing the number or proportion of cells of various cell types or cell states present in the sample, and wherein q satisfies the matrix equation Pq=m, where P is a matrix of pure cell type or pure cell state state signatures and m is a vector of quantities representing expression levels of genes included in the pure cell type or pure cell state signatures in the mixed cell population, and wherein one or more of the elements solved for represents the number, relative number, or proportion of cells that have responded to the treatment or stimulation.

33. The method of claim 32, wherein:
the computing step comprises computing a least squares solution.

34. The method of claim 32, wherein:
the elements of the vector m are RNA expression levels, which are optionally measured using a microarray, or wherein the elements of the vector m are protein levels.

35. A method of determining whether cells in a sample have been exposed to a condition comprising steps of:
providing a sample of cells, wherein some or all of the cells have been exposed to the condition, and wherein exposure to the condition leads to a change in expression of at least one gene;
providing or determining a pure cell type or pure cell state signature for cells of different cell types or cell states in the sample, wherein the pure cell type or pure cell state signatures include a cell type or cell state signature for a cell that has been exposed to the condition and a pure cell type or pure cell state signature for a cell that has not been exposed to the condition; and
quantitatively determining the number, relative number, or proportion of cells that have responded to the treatment using the pure cell type or pure cell state signatures.

36. The method of claim 35, wherein the quantitatively determining step comprises:
computing an approximate solution for one or more elements in a vector q, where q is a vector of quantities representing the number or proportion of cells of various types or states present in the sample, and wherein q satisfies the matrix equation Pq=m, where P is a matrix of pure cell type or pure cell state signatures and m is a vector of quantities representing expression levels of genes included in the pure cell type or pure cell state signatures in the mixed cell population, and wherein one or more of the elements solved for represents the number, relative number, or proportion of cells that have responded to the treatment.

37. The method of claim 36, wherein:
the computing step comprises computing a least squares solution.

38. The method of claim 36, wherein:
the elements of the vector m are RNA expression levels, which are optionally measured using a microarray, or wherein the elements of the vector m are protein levels.

39. A method for determining whether a difference in measured expression level of a gene in a plurality of samples reflects a difference in absolute expression of the one or more genes on a per cell basis or reflects a difference in number, relative number, or proportion of cells of different cell types or states in the samples, the method comprising steps of:
quantitatively determining the number, relative number, or proportion of cells of different cell types or cell states in two or more of the samples; and
determining, based on the number, relative number, or proportion of cells of different cell types or cell states, whether a difference in expression level of the gene between the samples reflects a difference in absolute expression on a per cell basis or a difference in the number, relative number, or proportion of cells of different cell types or cell states in the samples.

40. The method of claim 39, wherein the quantitatively determining step comprises performing a comparison of the number, relative number, or proportion of cells of different cell types or cell states in a plurality of samples, wherein a comparison indicating that the number, relative number, or proportion of cells of different cell types or cell states in any two or more samples are not substantially the same serves to indicate that any differences in expression of the gene arise at least in part as a result of differences in the number, relative number, or proportion of cells of different cell types or cell states in the samples, and a comparison indicating that the number, relative number, or proportion of cells of different cell types or cell states in any two or more samples are substantially the same serves to indicate that any differences in expression of the gene are actual changes in expression, and wherein one or more of the samples is optionally a reference or control sample.
